SEQUENCE LISTING (22 sequences)

SEQ ID NO: 1 - protein, 34 aa, Influenza A virus
LMSQSRTREILTKTTVDHMAIIKKYTSGRQEKNP

SEQ ID NO: 2 - protein, 26 aa, Influenza A virus
KVERLKHGTFGPVHFRNQVKIRRRVD

SEQ ID NO: 3 - protein, 43 aa, Influenza A virus
YIEVLHLTQGTCWEQMYTPGGEVRNDDVDQSLIIAARNIVRRA

SEQ ID NO: 4 - protein, 23 aa, Influenza A virus
LTGNLQTLKIRVHEGYEEFTMVG

SEQ ID NO: 5 - protein, 47 aa, Influenza A virus
VAMVFSQEDCMIKAVRGDLNFVNRANQRLNPMHQLLRHFQKDAKVLF

SEQ ID NO: 6 - protein, 32 aa, Influenza A virus
LTITYSSSMMWEINGPESVLVNTYQWIIRNWE

SEQ ID NO: 7 - protein, 21 aa, Influenza A virus
  VARIANT (18)...(18): Met or Leu
QSRMQFSSLTVNVRGSGMRIL

SEQ ID NO: 8 - protein, 51 aa, Influenza A virus
  VARIANT (12)...(12): Val or Ile
MDVNPTLLFLKVPAQNAISTTFPYTGDPPYSHGTGTGYTMDTVNRTHQYSE

SEQ ID NO: 9 - protein, 35 aa, Influenza A virus
VQQTRVDKLTQGRQTYDWTLNRNQPAATALANTIE

SEQ ID NO: 10 - protein, 24 aa, Influenza A virus
  VARIANT (3)...(3): Ile or Met
LSIAPIMFSNKMARLGKGYMFESK

SEQ ID NO: 11 - protein, 89 aa, Influenza A virus
  VARIANT (31)...(31): Ile or Val
TGTFEFTSFFYRYGFVANFSMELPSFGVSGINESADMSIGVTVIKNNMINNDLGPATAQMALQLFIKDYRYTYRCHRGDTQIQTRRSFE

SEQ ID NO: 12 - protein, 36 aa, Influenza A virus
MEYDAVATTHSWIPKRNRSILNTSQRGILEDEQMYQ

SEQ ID NO: 13 - protein, 26 aa, Influenza A virus
KIETNKFAAICTHLEVCFMYSDFHFI

SEQ ID NO: 14 - protein, 20 aa, Influenza A virus
YLEEHPSAGKDPKKTGGPIY

SEQ ID NO: 15 - protein, 30 aa, Influenza A virus
HENRMVLASTTAKAMEQMAGSSEQAAEAME

SEQ ID NO: 16 - protein, 607 aa, Artificial Sequence
  Influenza peptide catenate with GPGPG spacers
LMSQSRTREILTKTTVDHMAIIKKYTSGRQEKNPGPGPGKVERLKHGTFGPVHFRNQVKIRRRVDGPGPGYIEVLHLTQGTCWEQMYTPGGEVRNDDVDQSLIIAARNIVRRAGPGPGLTGNLQTLKIRVHEGYEEFTMVGGPGPGVAMVFSQEDCMIKAVRGDLNFVNRANQRLNPMHQLLRHFQKDAKVLFGPGPGLTITYSSSMMWEINGPESVLVNTYQWIIRNWEGPGPGQSRMQFSSLTVNVRGSGMRILGPGPGMDVNPTLLFLKVPAQNAISTTFPYTGDPPYSHGTGTGYTMDTVNRTHQYSEGPGPGVQQTRVDKLTQGRQTYDWTLNRNQPAATALANTIEGPGPGLSIAPIMFSNKMARLGKGYMFESKGPGPGTGTFEFTSFFYRYGFVANFSMELPSFGVSGINESADMSIGVTVIKNNMINNDLGPATAQMALQLFIKDYRYTYRCHRGDTQIQTRRSFEGPGPGMEYDAVATTHSWIPKRNRSILNTSQRGILEDEQMYQGPGPGKIETNKFAAICTHLEVCFMYSDFHFIGPGPGYLEEHPSAGKDPKKTGGPIYGPGPGHENRMVLASTTAKAMEQMAGSSEQAAEAME

SEQ ID NO: 17 - DNA, 1821 bp, Artificial Sequence
  encodes Influenza A peptide catenate with GPGPG spacers
ctgatgagccagagccggacccgggagatcctgaccaagaccaccgtggaccacatggccatcatcaagaagtacaccagcggcagacaggaaaagaaccccggacccggacctggcaaggtggagagactgaagcacggcaccttcggccccgtgcacttccggaaccaggtgaagatccggcggagagtggatggaccaggccctggctacatcgaggtgctgcacctgacccagggcacctgttgggagcagatgtacacccctggcggcgaagtgcggaacgacgacgtggaccagagcctgatcattgccgccagaaacatcgtgcggagagccggccctggacctggactgaccggcaacctgcagaccctgaagatcagagtgcacgagggctacgaagagttcaccatggtgggcggaccaggacctggcgtggccatggtgttcagccaggaagattgcatgatcaaggccgtgcggggcgacctgaacttcgtgaaccgggccaaccagcggctgaaccccatgcaccagctgctgcggcacttccagaaagacgccaaggtgctgtttggcccaggcccaggcctgaccatcacctacagcagcagcatgatgtgggagatcaacggccccgagagcgtgctggtgaacacctaccagtggatcatccggaactgggagggacctggccccggacagagccggatgcagttcagcagcctgaccgtgaatgtgcggggcagcggcatgagaatcctcggcccaggacccggcatggacgtgaaccccaccctgctgtttctgaaggtgcccgcccagaacgccatcagcaccaccttcccttacaccggcgaccctccctactctcacggcaccggcaccggctacaccatggacaccgtgaacagaacccaccagtacagcgaaggaccaggaccaggcgtgcagcagacccgggtggacaagctgacacagggccggcagacctacgactggaccctgaacagaaaccagcctgccgccaccgccctggccaataccatcgaaggccccggaccaggactgtctatcgcccccatcatgttcagcaacaagatggcccggctgggcaagggctacatgttcgagagcaaggggcctggaccaggcacaggcacctttgagttcaccagctttttctacagatacggcttcgtggccaacttcagcatggaactgcccagcttcggcgtgagcggcatcaacgagagcgccgacatgagcatcggcgtgaccgtgatcaagaacaacatgatcaacaacgacctgggacctgccacagctcagatggccctgcagctgttcatcaaggactaccggtacacctaccggtgccacagaggcgacacccagatccagaccaggcggagctttgagggcccagggccagggatggaatacgacgccgtggccaccacccacagctggatccccaagcggaaccggtccatcctgaacaccagccagcggggcatcctggaagatgaacagatgtaccaggggcctggacctggcaagatcgagacaaacaagttcgccgccatctgcacccacctggaagtgtgcttcatgtacagcgacttccacttcatcggacctggacccggctacctggaagaacaccccagcgccggcaaggaccctaagaaaaccggcggacccatctatggacctgggcctggccacgagaacagaatggtgctggcctctaccaccgccaaggccatggaacagatggccggcagcagcgaacaggccgccgaagccatggaa

SEQ ID NO: 18 - DNA, 1140 bp, Homo sapiens
  encodes Influenza A peptide catenate with GPGPGP spacers
atggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgtccagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagccggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagc

SEQ ID NO: 19 - protein, 380 aa, Homo sapiens
MAPRSARRPLLLLLLLLLLGLMHCASAAMFMVKNGNGTACIMANFSAAFSVNYDTKSGPKNMTLDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENS

SEQ ID NO: 20 - DNA, 117 bp, Homo sapiens
acgctgatccccatcgctgtgggtggtgccctggcggggctggtcctcatcgtcctcatcgcctacctcgtcggcaggaagaggagtcacgcaggctaccagactatctagggtacc

SEQ ID NO: 21 - protein, 37 aa, Homo sapiens
LIPIAVGGALAGLVLIVLIAYLVGRKRSHAGYQTIGT

SEQ ID NO: 22 - DNA, 7582 bp, Artificial Sequence
  p43-LAMP/FluVax with CMV promoter, chimeric intron, SV40 polyA signal, amplicilin resistance, ColE1, and f1(+) origin
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcatggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgtccagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagccggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcctcgagctgatgagccagagccggacccgggagatcctgaccaagaccaccgtggaccacatggccatcatcaagaagtacaccagcggcagacaggaaaagaaccccggacccggacctggcaaggtggagagactgaagcacggcaccttcggccccgtgcacttccggaaccaggtgaagatccggcggagagtggatggaccaggccctggctacatcgaggtgctgcacctgacccagggcacctgttgggagcagatgtacacccctggcggcgaagtgcggaacgacgacgtggaccagagcctgatcattgccgccagaaacatcgtgcggagagccggccctggacctggactgaccggcaacctgcagaccctgaagatcagagtgcacgagggctacgaagagttcaccatggtgggcggaccaggacctggcgtggccatggtgttcagccaggaagattgcatgatcaaggccgtgcggggcgacctgaacttcgtgaaccgggccaaccagcggctgaaccccatgcaccagctgctgcggcacttccagaaagacgccaaggtgctgtttggcccaggcccaggcctgaccatcacctacagcagcagcatgatgtgggagatcaacggccccgagagcgtgctggtgaacacctaccagtggatcatccggaactgggagggacctggccccggacagagccggatgcagttcagcagcctgaccgtgaatgtgcggggcagcggcatgagaatcctcggcccaggacccggcatggacgtgaaccccaccctgctgtttctgaaggtgcccgcccagaacgccatcagcaccaccttcccttacaccggcgaccctccctactctcacggcaccggcaccggctacaccatggacaccgtgaacagaacccaccagtacagcgaaggaccaggaccaggcgtgcagcagacccgggtggacaagctgacacagggccggcagacctacgactggaccctgaacagaaaccagcctgccgccaccgccctggccaataccatcgaaggccccggaccaggactgtctatcgcccccatcatgttcagcaacaagatggcccggctgggcaagggctacatgttcgagagcaaggggcctggaccaggcacaggcacctttgagttcaccagctttttctacagatacggcttcgtggccaacttcagcatggaactgcccagcttcggcgtgagcggcatcaacgagagcgccgacatgagcatcggcgtgaccgtgatcaagaacaacatgatcaacaacgacctgggacctgccacagctcagatggccctgcagctgttcatcaaggactaccggtacacctaccggtgccacagaggcgacacccagatccagaccaggcggagctttgagggcccagggccagggatggaatacgacgccgtggccaccacccacagctggatccccaagcggaaccggtccatcctgaacaccagccagcggggcatcctggaagatgaacagatgtaccaggggcctggacctggcaagatcgagacaaacaagttcgccgccatctgcacccacctggaagtgtgcttcatgtacagcgacttccacttcatcggacctggacccggctacctggaagaacaccccagcgccggcaaggaccctaagaaaaccggcggacccatctatggacctgggcctggccacgagaacagaatggtgctggcctctaccaccgccaaggccatggaacagatggccggcagcagcgaacaggccgccgaagccatggaagaattcacgctgatccccatcgctgtgggtggtgccctggcggggctggtcctcatcgtcctcatcgcctacctcgtcggcaggaagaggagtcacgcaggctaccagactatctagggtacctctagagtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgcag

(19) United States
(12) Patent Application Publication
(10) Pub. No.: US 2012/0294879 A1
(43) Pub. Date: Nov. 22, 2012

(54) CONSENSUS SEQUENCE FOR INFLUENZA A VIRUS

(75) Inventors: J. Thomas August, Baltimore, MD (US); Paul ThiamJoo Tan, Singapore (SG); Tin Wee Tan, Singapore (SG); Mohammad Asif Khan, Singapore (SG)

(73) Assignees: NATIONAL UNIVERSITY OF SINGAPORE, Singapore (SG); THE JOHNS HOPKINS UNIVERSITY, Baltimore, MD (US)

(21) Appl. No.: 13/501,339

(22) PCT Filed: Oct. 13, 2010
(86) PCT No.: PCT/US10/52432

§ 371 Date: Jul. 20, 2012

Related U.S. Application Data
(60) Provisional application No. 61/251,077, filed on Oct. 13, 2009.
(60) Provisional application No. 61/358,437, filed on Jun. 25, 2010.

(51) Int. Cl. A61K 39/145; C12N 15/33; C12N 15/85; C12N 15/86; C07K 14/11; C12P 21/02; C07K 7/06; A61P 31/16; C07K 19/00; C07K 7/08; C12N 15/62; C12N 5/10

(52) U.S. Cl. 424/186.1; 536/23.4; 536/23.72; 435/320.1; 435/366; 435/325; 435/69.3; 435/455; 530/328; 530/327; 530/326; 530/325; 530/324; 530/350

(57) ABSTRACT

Pandemic A(H1N1) continues its global spread, and vaccine production is a serious problem. Protection by current vaccines is limited by the mutational differences that rapidly accumulate in the circulating strains, especially in the virus surface proteins. New vaccine strategies are focusing at conserved regions of the viral internal proteins to produce T cell epitope-based vaccines. T cell responses have been shown to reduce morbidity and promote recovery in mouse models of influenza challenge. We previously reported 54 highly conserved sequences of NP, M1 and the polymerases of all human H1N1, H3N2, H1N2, and H5N1, and avian subtypes over the past 30 years. Sixty-three T cell epitopes elicited responses in HLA transgenic mice (A2, A24, B7, DR2, DR3 and DR4). These epitopes were compared to the 2007-2009 human H1N1 sequences to identify conserved and variant residues. Seventeen T cell epitopes of PB1, PB2, and M1 were selected as vaccine targets by analysis of sequence conservation and variability, functional avidity, non-identity to human peptides, clustered localization, and promiscuity to multiple HLA alleles. The vaccines composed of these epitopes, being highly conserved and temporally stable, would be useful for any avian or human influenza A virus.

This invention was made using funding from the U.S. government. Consequently, the U.S. government retains certain rights according to the terms of N01 AI-040085.

### TECHNICAL FIELD OF THE INVENTION

This invention is related to the area of influenza viruses. In particular, it relates to vaccines and constituents of vaccines.

### BACKGROUND OF THE INVENTION

Influenza A viruses are major pathogens of avian origin, affecting humans and other mammals, with global spread and rapid evolutionary mutational change. Of particular global concern are the several ways a human influenza pandemic could emerge. One is through the occurrence of a novel highly pathogenic zoonotic strain capable of infecting humans, such as the H5N1 avian pathogen that infected 436 humans with a 60% mortality rate (as of 1 Jul. 2009, WHO). Another possibility is through mutation from a mild to an increased pathogenic human transmissible strain, such as the current A(H1N1) pandemic. The most threatening is mutations giving rise to a new highly transmissible-and-pathogenic human strain where there is no human immunity, as occurred with the original 1918 Spanish influenza. In any event, history teaches us that a vaccine to prevent a new influenza A pandemic must be effective against all future forms of the virus.

Influenza A viruses are single stranded, negative-sense RNA viruses belonging to the family Orthomyxoviridae. The genome is composed of 8 RNA strands of about 13,500 bases, encoding at least ten viral proteins. The viral envelope is a lipid bilayer, consisting of the interior matrix protein 1 (M1) and three exterior transmembrane proteins: hemagglutinin (HA), neuraminidase (NA), and matrix protein 2 (M2). The viral core contains viral ribonucleoprotein complex particles, consisting of viral RNA, nucleoprotein (NP), and three polymerase proteins (PB1, PB2, and PA). Mutation in the viral RNA genome occurs by two mechanisms, known as antigenic drift and antigenic shift. Antigenic drift is the frequent occurrence of point mutations resulting from defects in RNA replication mechanisms, while antigenic shift is less frequent, involving re-assortment of the RNA segments arising from exchanges between different strains in host cells infected by multiple viruses.

Protection by current human influenza vaccines is achieved by use of inactivated or attenuated forms of the corresponding pathogen and appears to require the function of neutralizing antibodies against the external HA and NA glycoproteins. However, these glycoproteins mutate rapidly through antigenic drift and current vaccines become ineffective as mutational differences accumulate in the circulating strains. To overcome the antigenic variability of influenza external glycoproteins, new vaccine strategies are increasingly directed at conserved regions of the viral internal proteins for production of T cell epitope-based vaccines against all influenza A virus subtypes and to obviate the need for yearly vaccine update. Several animal model studies taking this approach have reported T cell responses that reduce morbidity and promote recovery in mouse models of influenza challenge [1-4]. Both CD8+ and CD4+ T cell responses are required; CD8+ T cells to kill infected cells [5,6] and CD4+ T cells for the development of an effective immune response and immune memory [7-9]. However, there is limited characterization of cellular viral antigens as vaccine targets. Very few human T cell epitopes of influenza proteins other than HA and NA are reported [10]. Moreover, even for the T cell epitope peptides that were identified, the actual epitope structures and the requirements of epitope amino- and carboxyl-termini for epitope processing and presentation in humans are for most, if not all, unknown.

We previously reported a detailed study of the evolutionarily conserved sequences of all human and avian influenza A viruses that were recorded over the past 30 years (36,343 sequences) [11]. Fifty-four (54) sequences of 9 or more amino acids of the PB2, PB1, PA, NP, and M1 sequences, conserved in at least 80%, and in most cases 95-100% of all recorded human H1N1, H3N2, H1N2, and H5N1, and avian subtypes were identified. These sequences have remained evolutionarily stable for all recorded influenza A viruses during the past decades, and are thus prime candidates for the development of T cell epitope-based vaccines against multiple influenza strains. However, the function of these conserved sequences as HLA-restricted T cell epitopes and the incidence of variant sequences in association with the conserved sequences were not known.

There is a continuing need in the art to identify and test influenza vaccines to reduce the incidence and/or severity of influenza A infections and/or pandemics.

### SUMMARY OF THE INVENTION

According to one aspect of the invention a polypeptide is provided. The polypeptide comprises: (a) a LAMP-1 lumenal sequence comprising SEQ ID NO: 19; (b) one or more segments of an influenza A protein, wherein said segments comprise at least 9 contiguous amino acid residues selected from SEQ ID NO: 1-15, wherein segments are linked together by 0-20 amino acid residues; and (c) a LAMP transmembrane and cytoplasmic tail comprising SEQ ID NO: 21, wherein the lumenal sequence is amino-terminal to the one or more segments of an influenza A protein which are amino-terminal to the LAMP transmembrane and cytoplasmic tail. The polypeptides may be combined to form compositions comprising a mixture of at least two polypeptides.

Other polypeptides which are provided include polypeptides consisting of an amino acid sequence selected from the group consisting of SEQ ID NO: 3, 4, 5, 6, 8, 11, and 12, as well as polypeptides which comprise less than a full-length PB1 or PB2 protein of influenza A virus and comprise an amino acid sequence selected from the group consisting of SEQ ID NO: 3, 4, 5, 6, 8, 11, and 12. The polypeptides may be combined to form compositions comprising a mixture of at least two polypeptides.

Another aspect of the invention is a polynucleotide which encodes a polypeptide. The polypeptide comprises: (a) a LAMP-1 lumenal sequence comprising SEQ ID NO: 19; (b) one or more segments of an influenza A protein, wherein said segments comprise at least 9 contiguous amino acid residues selected from SEQ ID NO: 1-15, wherein segments are linked together by 0-20 amino acid residues; and (c) a LAMP transmembrane and cytoplasmic tail comprising SEQ ID NO: 21, wherein the lumenal sequence is amino-terminal to the one or more segments of an influenza A protein which are amino-terminal to the LAMP transmembrane and cytoplasmic tail. Such polynucleotides can be combined to form mixtures of at least two polynucleotides.

Another aspect of the invention is a polynucleotide which encodes a polypeptide. The polypeptide consists of an amino acid sequence selected from the group consisting of SEQ ID NO: 3, 4, 5, 6, 8, 11, and 12, or the polypeptide comprises less than a full-length PB1 or PB2 protein of influenza A virus and comprise an amino acid sequence selected from the group consisting of SEQ ID NO: 3, 4, 5, 6, 8, 11, and 12. Such polynucleotides can be combined to form mixtures of at least two polynucleotides.

Yet another aspect of the invention is a nucleic acid vector that comprises the polynucleotide. The polynucleotide may encode a polypeptide which comprises: (a) a LAMP-1 lumenal sequence comprising SEQ ID NO: 19; (b) one or more segments of an influenza A protein, wherein said segments comprise at least 9 contiguous amino acid residues selected from SEQ ID NO: 1-15, wherein segments are linked together by 0-20 amino acid residues; and (c) a LAMP transmembrane and cytoplasmic tail comprising SEQ ID NO: 21, wherein the lumenal sequence is amino-terminal to the one or more segments of an influenza A protein which are amino-terminal to the LAMP transmembrane and cytoplasmic tail. Alternatively the polynucleotide may encode a polypeptide consisting of an amino acid sequence selected from the group consisting of SEQ ID NO: 3, 4, 5, 6, 8, 11, and 12, or it may encode a polypeptide which comprises less than a full-length PB1 or PB2 protein of influenza A virus and comprise an amino acid sequence selected from the group consisting of SEQ ID NO: 3, 4, 5, 6, 8, 11, and 12.

Still another aspect of the invention is a host cell. The host cell comprises the nucleic acid vector that comprises the polynucleotide that encodes a polypeptide. The polypeptide comprises: (a) a LAMP-1 lumenal sequence comprising SEQ ID NO: 19; (b) one or more segments of an influenza A protein, wherein said segments comprise at least 9 contiguous amino acid residues selected from SEQ ID NO: 1-15, wherein segments are linked together by 0-20 amino acid residues; and (c) a LAMP transmembrane and cytoplasmic tail comprising SEQ ID NO: 21, wherein the lumenal sequence is amino-terminal to the one or more segments of an influenza A protein which are amino-terminal to the LAMP transmembrane and cytoplasmic tail. Alternatively, the polypeptide consists of an amino acid sequence selected from the group consisting of SEQ ID NO: 3, 4, 5, 6, 8, 11, and 12, or the polypeptide comprises less than a full-length PB1 or PB2 protein of influenza A virus and comprise an amino acid sequence selected from the group consisting of SEQ ID NO: 3, 4, 5, 6, 8, 11, and 12.

According to another aspect of the invention a method is provided for producing a polypeptide. A host cell is cultured under conditions in which the host cell expresses a polypeptide. The polypeptide comprises: (a) a LAMP-1 lumenal sequence comprising SEQ ID NO: 19; (b) one or more segments of an influenza A protein, wherein said segments comprise at least 9 contiguous amino acid residues selected from SEQ ID NO: 1-15, wherein segments are linked together by 0-20 amino acid residues; and (c) a LAMP transmembrane and cytoplasmic tail comprising SEQ ID NO: 21, wherein the lumenal sequence is amino-terminal to the one or more segments of an influenza A protein which are amino-terminal to the LAMP transmembrane and cytoplasmic tail. Alternatively, the polypeptide consists of an amino acid sequence selected from the group consisting of SEQ ID NO: 3, 4, 5, 6, 8, 11, and 12, or the polypeptide comprises less than a full-length PB1 or PB2 protein of influenza A virus and comprise an amino acid sequence selected from the group consisting of SEQ ID NO: 3, 4, 5, 6, 8, 11, and 12.

Another aspect of the invention is a method of producing a cellular vaccine. An antigen presenting cell is transfected with a nucleic acid vector which comprises a polynucleotide which encodes a polypeptide. The antigen presenting cells thereafter express the polypeptide. The polypeptide comprises: (a) a LAMP-1 lumenal sequence comprising SEQ ID NO: 19; (b) one or more segments of an influenza A protein, wherein said segments comprise at least 9 contiguous amino acid residues selected from SEQ ID NO: 1-15, wherein segments are linked together by 0-20 amino acid residues; and (c) a LAMP transmembrane and cytoplasmic tail comprising SEQ ID NO: 21, wherein the lumenal sequence is amino-terminal to the one or more segments of an influenza A protein which are amino-terminal to the LAMP transmembrane and cytoplasmic tail. Alternatively, the polypeptide consists of an amino acid sequence selected from the group consisting of SEQ ID NO: 3, 4, 5, 6, 8, 11, and 12, or the polypeptide comprises less than a full-length PB1 or PB2 protein of influenza A virus and comprise an amino acid sequence selected from the group consisting of SEQ ID NO: 3, 4, 5, 6, 8, 11, and 12.

An additional aspect of the invention is a method of making a vaccine. A polypeptide and an immune adjuvant are mixed together. The polypeptide comprises: (a) a LAMP-1 lumenal sequence comprising SEQ ID NO: 19; (b) one or more segments of an influenza A protein, wherein said segments comprise at least 9 contiguous amino acid residues selected from SEQ ID NO: 1-15, wherein segments are linked together by 0-20 amino acid residues; and (c) a LAMP transmembrane and cytoplasmic tail comprising SEQ ID NO: 21, wherein the lumenal sequence is amino-terminal to the one or more segments of an influenza A protein which are amino-terminal to the LAMP transmembrane and cytoplasmic tail. Alternatively, the polypeptide consists of an amino acid sequence selected from the group consisting of SEQ ID NO: 3, 4, 5, 6, 8, 11, and 12, or the polypeptide comprises less than a full-length PB1 or PB2 protein of influenza A virus and comprise an amino acid sequence selected from the group consisting of SEQ ID NO: 3, 4, 5, 6, 8, 11, and 12.

A further aspect of the invention is a vaccine composition which comprises a polypeptide. The polypeptide comprises: (a) a LAMP-1 lumenal sequence comprising SEQ ID NO: 19; (b) one or more segments of an influenza A protein, wherein said segments comprise at least 9 contiguous amino acid residues selected from SEQ ID NO: 1-15, wherein segments are linked together by 0-20 amino acid residues; and (c) a LAMP transmembrane and cytoplasmic tail comprising SEQ ID NO: 21, wherein the lumenal sequence is amino-terminal to the one or more segments of an influenza A protein which are amino-terminal to the LAMP transmembrane and cytoplasmic tail. Alternatively, the polypeptide consists of an amino acid sequence selected from the group consisting of SEQ ID NO: 3, 4, 5, 6, 8, 11, and 12, or the polypeptide comprises less than a full-length PB1 or PB2 protein of influenza A virus and comprise an amino acid sequence selected from the group consisting of SEQ ID NO: 3, 4, 5, 6, 8, 11, and 12.

A further aspect of the invention is a method of immunizing a human or other animal subject. A polypeptide or a nucleic acid vector or a host cell is administered to the human or other animal subject in an amount effective to elicit influenza A-specific T cell activation. The polypeptide comprises: comprises: (a) a LAMP-1 lumenal sequence comprising SEQ ID NO: 19; (b) one or more segments of an influenza A protein, wherein said segments comprise at least 9 contiguous amino acid residues selected from SEQ ID NO: 1-15, wherein segments are linked together by 0-20 amino acid residues; and (c) a LAMP transmembrane and cytoplasmic tail comprising SEQ ID NO: 21, wherein the lumenal sequence is amino-terminal to the one or more segments of an influenza A protein which are amino-terminal to the LAMP transmembrane and cytoplasmic tail. Alternatively, the polypeptide consists of an amino acid sequence selected from the group consisting of SEQ ID NO: 3, 4, 5, 6, 8, 11, and 12, or the polypeptide comprises less than a full-length PB1 or PB2 protein of influenza A virus and comprise an amino acid sequence selected from the group consisting of SEQ ID NO: 3, 4, 5, 6, 8, 11, and 12.

These and other embodiments which will be apparent to those of skill in the art upon reading the specification provide the art with

### BRIEF DESCRIPTION OF THE DRAWINGS

FIG. 1 shows localization of HLA-restricted T-cell epitopes of conserved sequences of influenza polymerases, NP, and M1 proteins. Numbers represent amino acid positions. Highly conserved amino acids are shown as grey boxes. T cell epitopes were restricted by HLA-DR4 (black boxes), -DR3 (blue boxes), -DR2 (brown boxes), -A24 (green boxes), and -B7 (orange boxes).

FIG. 2 shows predicted HLA-supertype-restricted T-cell epitopes of conserved sequences of influenza PB2, PB1, PA, NP, and M1 proteins.

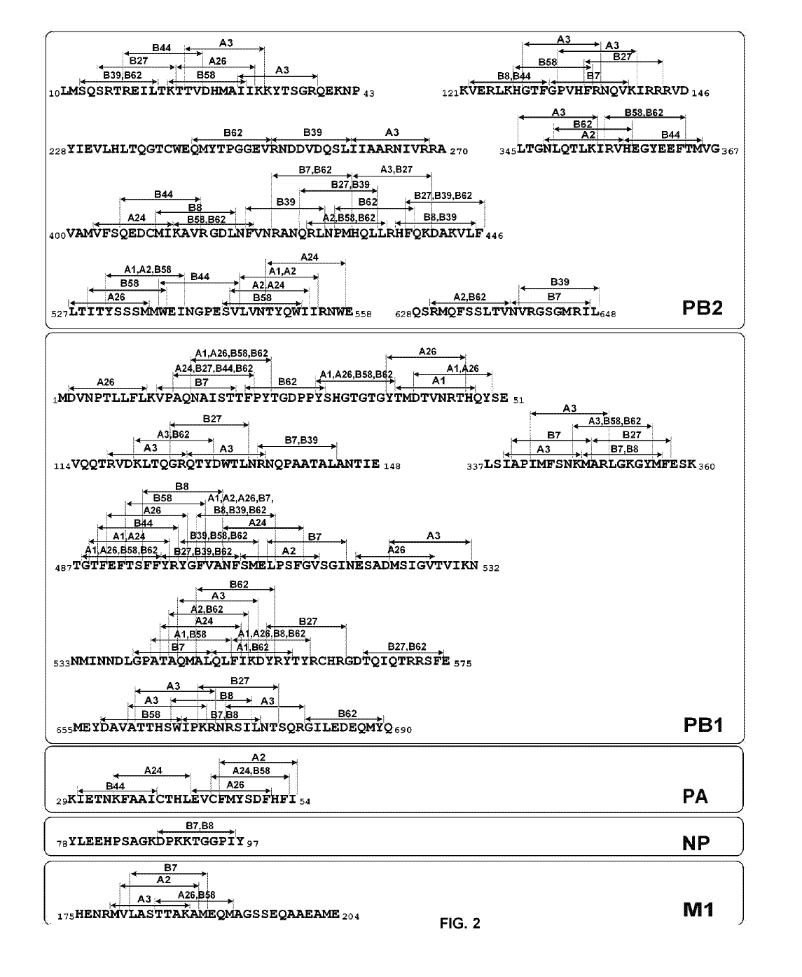

### DETAILED DESCRIPTION OF THE INVENTION

The inventors have identified and characterized peptide segments of influenza virus A/New York/348/2003 (H1N1) that contain conserved sequences and elicit HLA-restricted T cell responses. HLA transgenic mice (HLA-A2, -A24, -B7, -DR2, -DR3 and -DR4) were immunized with selected peptides. The peptides that elicited T cell activation by IFN-γ ELISpot assay and thus functioned as human T cell epitope peptides were selected and analyzed for properties relevant in vaccine development. The evolutionary variability and the relationship of the 2003 H1N1 T cell epitope peptide sequences to the corresponding 2007-2009 human H1N1 sequences were studied. The results identified (i) the H1N1 HLA-restricted T cell epitope peptides in the context of pathogenic influenza A conserved sequences and (ii) the variant amino acids (aa) and percentage representation of 2007-2009 H1N1 strains as compared to the 2003 A/New York/348 strain.

At least 9, 11, 13, 15, 17, 19, 20, or 21 amino acids of at least two of peptide segments identified as highly conserved and highly non-variant can optionally be linked together using 0-20 amino acids residues, such as GPGPG (alternating glycine and proline residue) linkers. Where distances between conserved sequences are small (one or two residues) and not highly variant, one may optionally join the sequences together with a natural but non-conserved amino acid or two, making larger mostly conserved segments. The linked segments may be from the same peptide segment or from different peptide segments. They may be from the same viral protein or from different viral proteins. The segments are shown in SEQ ID NO: 1-15. The linked segments form a catenate. The catenate may be flanked by two portions of the human LAMP-1 protein, also known as CD107a. The N-terminal portion is the luminal portion of the LAMP-1 protein. The C-terminal portion is the transmembrane domain and the short cytoplasmic tail. Thus the segment or the catenate is inserted in the midst of the LAMP-1 protein forming a chimeric protein. The chimeric protein may comprise at least 9 amino acids of at least 2, 3, 4, 5, 6, 7, 8, 9, 10, 11, 12, 13, 14, or 15 of the peptide segments. If duplicates are used or more than one of the at least 9-amino acid stretches from a single peptide segment are used, then more than 15 of the at least 9-amino acid stretches may be in the catenate. LAMP-1 chimeric proteins are used for antigen processing and presentation to the immune systems.

The polypeptides need not be in catenates and need not be in LAMP-1 chimeric proteins. The polyepeptides may be isolated and consist of a segment as shown in SEQ ID NO:1-15, such as any of SEQ ID NO:3, 4, 5, 6, 8, 11, and 12. Such polyeptides may be made synthetically or recombinantly. They may be isolated from natural sources and enzymatically digested and purified. Any manner of making them as is known in the art may be used. Typically the polypeptides are less than full-length influenza proteins. In the case of PB1 and PB2 polypeptides, the polypeptides are less than 150, less than 125, less than 100, less than 75, or less than 50 amino acid residues of PB 1 or PB2 in length. The polypeptides may also comprise other amino acid sequences linked to the influenza sequences. The linked sequences may be selected, e.g., to facilitate processing or production. The linked sequences may be used to improve physiological processing, like the LAMP-1 sequences. The sequences may be used to improve presentation to the immune system.

An alternative to catenates is mixtures of polypeptides (or polynucleotides encoding them). The mixtures may comprise at least 2, 3, 4, 5, 6, 7, 8, 9, 10, 11, 12, 13, 14, or 15 of the polypeptides of SEQ ID NO: 1-15. The mixtures may also comprise immune adjuvants, as are known in the art.

Any linkers may be used between influenza polypeptides in catenates. They may have glycine and proline residues in a different pattern than alternating. They may have a different length of glycine and proline residues. Linkers with other natural or non-naturally occurring amino acid residues may be used. Particular properties may be imparted by the linkers. They may provide a particular structure or property, for example a particular kink or a particular cleavable site. Design is within the skill of the art.

Polynucleotides which encode the polyeptides or chimeric proteins may be designed and made by techniques well known in the art. The natural sequences used by influenza virus A may be used. Alternatively non-natural sequences may be used, including in one embodiment, sequences that are codon-optimized for humans. Design of human codon optimized sequences is well within the skill of the ordinary artisan. Data regarding the most frequently used codons in the human genome are readily available. Optimization may be applied partially or completely.

The polynucleotides which encode the polyeptides or chimeric proteins can be replicated and/or expressed in vectors, such as DNA virus vectors, RNA virus vectors, and plasmid vectors. Preferably these will contain promoters for expressing the polyeptides or chimeric proteins in human or other mammalian or other animal cells. An example of a suitable promoter is the cytomegalovirus (CMV) promoter. Promoters may be inducible or repressible. They may be constitutive. They may express at high or low levels, as desired in a particular application. The vectors may be propagated in host cells for expression and collection of chimeric protein. Suitable vectors will depend on the host cells selected. In one embodiment host cells are grown in culture and the polypeptide is harvested from the cells or from the culture medium. Suitable purification techniques can be applied to the polyeptides or chimeric proteins as are known in the art. In another embodiment one transfects antigen presenting cells for ultimate delivery to a vaccinee of a cellular vaccine which expresses and presents antigen to the vaccinee. Suitable antigen presenting cells include dendritic cells, B cells, macrophages, and epithelial cells. In another embodiment vectors are directly administered to a vaccinee for expression in the vaccinee.

Immune adjuvants may be administered with the vaccines of the present invention, whether the vaccines are polypeptides, polynucleotides, nucleic acid vectors, or cellular vaccines. The adjuvants may be mixed with the specific vaccine substance prior to administration or may be delivered separately to the recipient, either before, during, or after the vaccine substance is delivered. Vaccines may be produced in any suitable manner, including in cells, in eggs, and synthetically. In addition to adjuvants, booster doses may be provided. Boosters may be the same or a complementary type of vaccine. Boosters may include a conventional live or attenuated influenza A viral vaccine. Typically a high titer of T cell activation and/or antibody is desired with a minimum of adverse side effects.

Any of the conventional or esoteric modes of administration may be used, including oral, mucosal, or nasal. Additionally intramuscular, intravenous, intradermal, or subcutaneous delivery may be used. The administration efficiency may be enhanced by using electroporation. Optimization of the mode of administration for the particular vaccine composition may be desirable.

Whole virus, including live, attenuated, or genetically inactivated, may be used as a booster or adjuvant. The virus may be administered at the same time as, before, after, or mixed with the polypeptide or polynucleotide vaccines.

An enigma of the immunobiology of influenza A is that vaccines fail to provide long term protection against infection and natural infection does not prevent reinfection. The rapid mutation of the viral proteins, particularly the external HA and NA proteins that are targets for neutralizing antibodies, is credited with a significant role in this loss of immunity. Defective adaptive immunity is also observed with several RNA viruses (including HIV-1 and dengue viruses) with high rates of mutation that result in multiple genetic variants bearing mutated T cell epitope sequences. This has resulted in widespread attention to the use of T cell epitopes incorporating conserved sequences of non-structural viral internal proteins [25-28]. However, the occurrence of reinfection, despite the human T cell response to conserved sequences after natural infection, suggests the function of a viral mechanism that intervenes in the host immune response to influenza infection. One possibility is the dual immunosuppressor roles of the influenza A NS 1 protein that inhibit innate immunity by preventing type I IFN release, as well as adaptive immunity by attenuating human dendritic cell maturation and the capacity of dendritic cells to induce T cell responses [29]. There is also the concept of immunological “original sin” where mutations in or adjacent to T cell epitopes preserve binding to MHC molecules but present an altered surface to the T-cell antigen receptor, resulting in an impaired or modified T cell response, including T cell immunosuppression [30-36].

In the examples shown below, HLA transgenic mice, HLA-A2, -A24, -B7, -DR2, -DR3 and DR4, were immunized with 196 overlapping H1N1 peptides of the A/New York/348/2003 strain that contained the highly conserved sequences of the M1, NP, PB1, PB2, and PA proteins of all reported human and avian influenza A viruses of the past 30 years [11]. Fifty-four (54) of these peptides (22 PB1, 16 PB2, 9 NP, 4 PA, and 3 M1) elicited 63 HLA-restricted T cell responses by IFN-γ ELISpot assay, where 7 peptides were restricted by multiple alleles. Further, the conserved T cell epitope peptides contained reported human T cell epitopes shared among pathogenic H1N1, H3N2 and H5N1 viral strains and were restricted by a broad range of HLA class I and II alleles. Thus, it is reasonable to expect that the conserved peptides identified here can elicit human T cell epitope responses in the context of several HLA alleles and HLA-supertypes [37] and that the memory T cells can cross-react with epitopes from H1N1, H3N2, and H5N1 [26,38,39]. The class I alleles described herein HLA-A*0201, -A*2402 and -B*0702 belong to the distinct supertypes A2, A24 and B7, respectively [40,41]. HLA class II supertypes are not as well documented but the 3 alleles of the transgenic mice of this study are assigned to supertypes DR1, DR3 and DR4 [42] based on similar protein and three-dimensional structures.

Analysis of the conservation and mutational variants of these H1N1 HLA-restricted epitope peptides revealed the marked effect that single aa mutations may have on the representation of T cell epitope peptides in evolving virus populations. Over the 3 years interval (2007 to 2009) between the database records analyzed by Heiny et al. (2006) to the current 2009 H1N1 sequence analysis, only 8 of the 54 highly conserved T cell epitope peptide sequences were without mutational change. These 8 peptides (M1175-191, 181-197, PB131-47, 120-136, 126-142, 489-505, 495-511, and 548-564) were representative of almost complete conservation, 95-100%, during the previous recorded history of human H1N1 virus sequences. All others of the identified HLA-restricted T cell epitope peptides contained at least 1 aa substitution, primarily but not exclusively, of the non-conserved aa of the H1N1 peptides. Our data suggest that the most favorable sequences for a T cell epitope-based vaccine are the 17 H1N1 T cell epitope peptides of the PB1, PB2, and M1 proteins (Table 6A). These were highly conserved over the 33 years (1977-2009) of the examined database records, representing 88 to 100% of all recorded avian and human influenza A viruses, including the H1N1 isolates. These 17 T cell epitopes are clustered and have distinct advantages in the design of an epitope-based genetic vaccine, including the retention of native sequences for the function of transporters associated with antigen processing (TAPs) [43] and for the flanking sequences that are reported to modulate epitope processing and function in the selection of immunodominant epitopes [44]. Each of these 17 sequences, except M1181-197 and PB1537-553, was also characterized by high apparent functional avidity at the lowest peptide concentration of 0.1 μg/ml in the IFN-γ ELISpot assay. Several studies showed that high avidity CD8+ T-cells were more effective in limiting viral replication in vitro [45-47]. Further, the 17 T-cell epitope peptides had no identity of 8 or more continuous aa to human peptides that might trigger onset of human autoimmune diseases. It is also noteworthy that several of the epitope peptides are located in described functional domains: PB1518-575 in the interacting domain of PB1 with PB2 (PB1506-659) [48]; and the overlapping PB2126-142 and PB2132-148 in the PB1- and NP-binding domain of PB21-269 [49]. T cell epitopes within functional domains would remain conserved over time as viral mutations useful towards immune escape may disrupt the function of the domains. Thus, a vaccine comprising these 17 highly conserved T cell epitope peptides, could greatly reduce, if not eliminate, the incidence of variant amino acids of the corresponding T cell epitopes of any future influenza A pathogen.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Materials and Methods

### Ethics Statement

Mice were maintained in a pathogen-free facility at the Johns Hopkins University according to IACUC guidelines.

### Influenza Peptides

Peptide arrays of PB2 (BEI Cat.: NR-2616), PB1 (NR-2617), PA (NR-2618), NP (NR-2611), and Ml (NR-2613) of influenza virus A/New York/348/2003 (H1N1) were obtained through the NIH Biodefense and Emerging Infections Research Resources Repository, NIAID, NIH (BEI). A total of 196 peptides (all 17 aa long) were selected to fully cover all highly conserved sequences under study. Where these sequences spanned two or more 17 aa peptides, the consecutive peptides overlapped by 11 aa. Two immunization peptide pools for immunization were formed: one composed of 84 PB2 and 13 M1 peptides (Table 1), and the second composed of 48 PB1, 23 PA, and 28 NP peptides (Table 2). Each of the 196 peptides was dissolved in 100% DMSO and constituted to 20% with sterile filtered water. The final concentration of each peptide was 2 μg/μl. The dissolved peptides were stored at −20° C.

TABLE 1
The first immunization peptide pool
consisted of 13 M1 and 84 PB2 peptides
of A/New York/348/2003 (H1N1) containing
the highly conserved aa (boldface).

| Protein | | | |
|---|---|---|---|
| | Peptides | | |
| M1 | 1 | MSLLTEVETYVLSIVPS | 17 |
| | 7 | VETYVLSIVPSGPLKAE | 23 |
| | 115 | IALSYSAGALASCMGLI | 131 |
| | 121 | AGALASCMGLIYNRMGA | 137 |
| | 127 | CMGLIYNRMGAVTTESA | 143 |
| | 169 | TNPLIRHENRMVLASTT | 185 |
| | 175 | HENRMVLASTTAKAMEQ | 191 |
| | 181 | LASTTAKAMEQMAGSSE | 197 |
| | 187 | KAMEQMAGSSEQAAEAM | 203 |
| | 193 | AGSSEQAAEAMEVASQA | 209 |
| | 199 | AAEAMEVASQARQMVQA | 215 |
| | 205 | VASQARQMVQAMRAIGT | 221 |
| | 210 | RQMVQAMRAIGTHPSSS | 226 |
| PB2 | 1 | MERIKELRNLMSQSRTR | 17 |
| | 7 | LRNLMSQSRTREILTKT | 23 |
| | 12 | SQSRTREILTKTTVDHM | 28 |
| | 18 | EILTKTTVDHMAIIKKY | 34 |
| | 24 | TVDHMAIIKKYTSGRQE | 40 |
| | 30 | IIKKYTSGRQEKNPSLR | 46 |
| | 36 | SGRQEKNPSLRMKWMMA | 52 |
| | 42 | NPSLRMKWMMAMKYPIT | 58 |
| | 48 | KWMMAMKYPITADKRIT | 64 |
| | 54 | KYPITADKRITEMIPER | 70 |
| | 60 | DKRITEMIPERNEQGQT | 76 |
| | 66 | MIPERNEQGQTLWSKVN | 82 |
| | 72 | EQGQTLWSKVNDAGSDR | 88 |
| | 78 | WSKVNDAGSDRVMISPL | 94 |
| | 84 | AGSDRVMISPLAVTWWN | 100 |
| | 90 | MISPLAVTWWNRNGPVA | 106 |
| | 96 | VTWWNRNGPVANTIHYP | 112 |
| | 102 | NGPVANTIHYPKIYKTY | 118 |
| | 108 | TIHYPKIYKTYFEKVER | 124 |
| | 114 | IYKTYFEKVERLKHGTF | 130 |
| | 120 | EKVERLKHGTFGPVHFR | 136 |
| | 126 | KHGTFGPVHFRNQVKIR | 142 |
| | 132 | PVHFRNQVKIRRRVDIN | 148 |
| | 137 | NQVKIRRRVDINPGHAD | 153 |
| | 143 | RRVDINPGHADLSAKEA | 159 |
| | 215 | TRFLPVAGGTSSVYIEV | 231 |
| | 221 | AGGTSSVYIEVLHLTQG | 237 |
| | 227 | VYIEVLHLTQGTCWEQM | 243 |
| | 233 | HLTQGTCWEQMYTPGGE | 249 |
| | 239 | CWEQMYTPGGEVRNDDV | 255 |
| | 245 | TPGGEVRNDDVDQSLII | 261 |
| | 251 | RNDDVDQSLIIAARNIV | 267 |
| | 256 | DQSLIIAARNIVRRAAV | 272 |
| | 262 | AARNIVRRAAVSADPLA | 278 |
| | 268 | RRAAVSADPLASLLEM | 283 |
| | 273 | SADPLASLLEMCHSTQI | 289 |
| | Sequences | | |
| | 279 | SLLEMCHSTQIGGTRMV | 295 |
| | 285 | HSTQIGGTRMVDILRQN | 301 |
| | 339 | KREEEVLTGNLQTLKLT | 355 |
| | 345 | LTGNLQTLKLTVHEGYE | 361 |
| | 351 | TLKLTVHEGYEEFTMVG | 367 |
| | 357 | HEGYEEFTMVGKRATAI | 373 |
| | 363 | FTMVGKRATAILRKATR | 379 |
| | 369 | RATAILRKATRRLIQLI | 385 |
| | 393 | SIVEAIVVAMVFSQED | 408 |
| | 398 | IVVAMVFSQEDCMVKAV | 414 |
| | 404 | FSQEDCMVKAVRGDLNF | 420 |
| | 410 | MVKAVRGDLNFVNRANQ | 426 |
| | 416 | GDLNFVNRANQRLNPMH | 432 |
| | 422 | NRANQRLNPMHQLLRHF | 438 |
| | 428 | LNPMHQLLRHFQKDAKV | 444 |
| | 434 | LLRHFQKDAKVLFLNWG | 450 |
| | 440 | KDAKVLFLNWGIEHIDN | 456 |
| | 458 | MGMIGILPDMTPSTEMS | 474 |
| | 464 | LPDMTPSTEMSMRGVRV | 480 |
| | 470 | STEMSMRGVRVSKMGVD | 486 |
| | 476 | RGVRVSKMGVDEYSNAE | 492 |
| | 482 | KMGVDEYSNAERVVVSI | 498 |
| | 500 | RFLRVRDQRGNVLLSPE | 516 |
| | 506 | DQRGNVLLSPEEVSETQ | 522 |
| | 512 | LLSPEEVSETQGTEKLT | 528 |
| | 518 | VSETQGTEKLTITYSSS | 534 |
| | 524 | TEKLTITYSSSMMWEIN | 540 |
| | 530 | TYSSSMMWEINGPESVL | 546 |
| | 536 | MWEINGPESVLINTYQW | 552 |
| | 542 | PESVLINTYQWIIRNWE | 558 |
| | 548 | NTYQWIIRNWETVKIQW | 564 |
| | 554 | IRNWETVKIQWSQNPTM | 570 |
| | 560 | VKIQWSQNPTMLYNKME | 576 |
| | 565 | SQNPTMLYNKMEFEPFQ | 581 |
| | 571 | LYNKMEFEPFQSLVPKA | 587 |
| | 577 | FEPFQSLVPKAIRGQYS | 593 |
| | 606 | VLGTFDTTQIIKLLPFA | 622 |
| | 612 | TTQIIKLLPFAAAPPKQ | 628 |
| | 618 | LLPFAAAPPKQSRMQFS | 634 |
| | 624 | APPKQSRMQFSSLTVNV | 640 |
| | 630 | RMQFSSLTVNVRGSGMR | 646 |
| | 636 | LTVNVRGSGMRILVRGN | 652 |
| | 642 | GSGMRILVRGNSPVFNY | 658 |
| | 678 | DPDEGTAGVESAVLRGF | 694 |
| | 684 | AGVESAVLRGFLILGKE | 700 |
| | 690 | VLRGFLILGKEDRRYGP | 706 |
| | 696 | ILGKEDRRYGPALSINE | 712 |
| | 702 | RRYGPALSINELSNLAK | 718 |

TABLE 2
The second immunization peptide pool
consisted of 28 NP, 23 PA and 48 PB1
peptides of A/New York/348/2003 (H1N1)
containing the highly conserved aa
(boldface).

| Protein | Sequences | | |
|---|---|---|---|
| NP | 1 | MASQGTKRSYEQMETDG | 17 |
| | 7 | KRSYEQMETDGERQNAT | 23 |
| | 25 | IRASVGRMIGGIGRFYI | 41 |
| | 31 | RMIGGIGRFYIQMCTEL | 47 |
| | 37 | GRFYIQMCTELKLNDYE | 53 |
| | 43 | MCTELKLNDYEGRLIQN | 59 |
| | 61 | LTIERMVLSAFDERRNK | 77 |
| | 67 | VLSAFDERRNKYLEEHP | 83 |
| | 73 | ERRNKYLEEHPSAGKDP | 89 |
| | 79 | LEEHPSAGKDPKKTGGP | 95 |
| | 85 | AGKDPKKTGGPIYKRVD | 101 |
| | 91 | KTGGPIYKRVDGKWVRE | 107 |
| | 103 | KWVRELVLYDKEEIRRI | 119 |
| | 109 | VLYDKEEIRRIWRQANN | 125 |
| | 115 | EIRRIWRQANNGDDATA | 131 |
| | 121 | RQANNGDDATAGLTHIM | 137 |
| | 127 | DDATAGLTHIMIWHSNL | 143 |
| | 133 | LTHIMIWHSNLNDTTYQ | 149 |
| | 139 | WHSNLNDTTYQRTRALV | 155 |
| | 234 | AQKAMMDQVRESRNPGN | 250 |
| | 240 | DQVRESRNPGNAEIEDL | 256 |
| | 246 | RNPGNAEIEDLTFLARS | 262 |
| | 402 | SAGQISTQPTFSVQRNL | 418 |
| | 408 | TQPTFSVQRNLPFDKTT | 424 |
| | 414 | VQRNLPFDKTTIMAAFT | 430 |
| | 450 | SARPEEVSFQGRGVFEL | 466 |
| | 456 | VSFQGRGVFELSDERAT | 472 |
| | 462 | GVFELSDERATNPIVPS | 478 |
| PA | 24 | YGEDLKIETNKFAAICT | 40 |
| | 30 | IETNKFAAICTHLEVCF | 46 |
| | 36 | AAICTHLEVCFMYSDFH | 52 |
| | 42 | LEVCFMYSDFHFINEQG | 58 |
| | 48 | YSDFHFINEQGESIIVE | 64 |
| | 120 | IGVTRREVHIYYLEKAN | 136 |
| | 126 | EVHIYYLEKANKIKSEK | 142 |
| | 132 | LEKANKIKSEKTHIHIF | 148 |
| | 138 | IKSEKTHIHIFSFTGEE | 154 |
| | 144 | HIHIFSFTGEEMATKAD | 160 |
| | 150 | FTGEEMATKADYTLDEE | 166 |
| | 179 | RQEMASRGLWDSFRQSE | 195 |
| | 185 | RGLWDSFRQSERGEETI | 201 |
| | 191 | FRQSERGEETIEERFEI | 207 |
| | 197 | GEETIEERFEITGTLRR | 213 |
| | 292 | IEDPNHEGEGIPLYDAI | 308 |
| | 298 | EGEGIPLYDAIKCMRTF | 314 |
| | 304 | LYDAIKCMRTFFGWKEP | 320 |
| | 404 | SSWIQNEFNKACELTDS | 420 |
| | 410 | EFNKACELTDSIWIELD | 426 |
| | 552 | SAIGQVSRPMFLYVRTN | 568 |
| | 558 | SRPMFLYVRTNGTSKIK | 574 |
| | 564 | YVRTNGTSKIKMKWGME | 580 |
| PB1 | 1 | MDVNPTLLFLKVPAQNA | 17 |
| | 7 | LLFLKVPAQNAISTTFP | 23 |
| | 13 | PAQNAISTTFPYTGDPP | 29 |
| | 19 | STTFPYTGDPPYSHGTG | 35 |
| | 25 | TGDPPYSHGTGTGYTMD | 41 |
| | 31 | SHGTGTGYTMDTVNRTH | 47 |
| | 37 | GYTMDTVNRTHQYSERG | 53 |
| | 43 | VNRTHQYSERGRWTKNT | 59 |
| | 108 | IETMEVVQQTRVDKLTQ | 124 |
| | 114 | VQQTRVDKLTQGRQTYD | 130 |
| | 120 | DKLTQGRQTYDWTLNRN | 136 |
| | 126 | RQTYDWTLNRNQPAATA | 142 |
| | 132 | TLNRNQPAATALANTIE | 148 |
| | 138 | PAATALANTIEVFRSNG | 154 |
| | 191 | VRDNVTKKMVTQRTIGK | 207 |
| | 197 | KKMVTQRTIGKKKHKLD | 213 |
| | 203 | RTIGKKKHKLDKRSYLI | 219 |
| | 328 | NQPEWFRNILSIAPIMF | 344 |
| | 334 | RNILSIAPIMFSNKMAR | 350 |
| | 340 | APIMFSNKMARLGKGYM | 356 |
| | 346 | NKMARLGKGYMFESKSM | 362 |
| | 352 | GKGYMFESKSMKLRTQI | 368 |
| | 358 | ESKSMKLRTQIPAEMLA | 374 |
| | 364 | LRTQIPAEMLANIDLKY | 380 |
| | 465 | RFYRTCKLLGINMSKKK | 481 |
| | 471 | KLLGINMSKKKSYINRT | 487 |
| | 477 | MSKKKSYINRTGTFEFT | 493 |
| | 483 | YINRTGTFEFTSFFYRY | 499 |
| | 489 | TFEFTSFFYRYGFVANF | 505 |
| | 495 | FFYRYGFVANFSMELPS | 511 |
| | 501 | FVANFSMELPSFGVSGV | 517 |
| | 507 | MELPSFGVSGVNESADM | 523 |
| | 513 | GVSGVNESADMSIGVTV | 529 |
| | 519 | ESADMSIGVTVIKNNMI | 535 |
| | 525 | IGVTVIKNNMINNDLGP | 541 |
| | 531 | KNNMINNDLGPATAQMA | 547 |
| | 537 | NDLGPATAQMALQLFIK | 553 |
| | 543 | TAQMALQLFIKDYRYTY | 559 |
| | 548 | LQLFIKDYRYTYRCHRG | 564 |
| | 554 | DYRYTYRCHRGDTQIQT | 570 |
| | 560 | RCHRGDTQIQTRRSFEI | 576 |
| | 566 | TQIQTRRSFEIKKLWDQ | 582 |
| | 650 | GPAKNMEYDAVATTHSW | 666 |
| | 656 | EYDAVATTHSWVPKRNR | 672 |
| | 662 | TTHSWVPKRNRSILNTS | 678 |
| | 668 | PKRNRSILNTSQRGILE | 684 |
| | 674 | ILNTSQRGILEDEQMYQ | 690 |
| | 680 | RGILEDEQMYQRCCNLF | 696 |

### HLA Transgenic Mice

Six different strains of HLA transgenic mice were used to cover HLA alleles of class I and class II supertypes. The HLA class I supertypes studied were HLA-A2 (A*0201) mice expressing a chimeric heavy chain with murine α3 domain and human β2m. Both H-2Db and murine β2m genes were disrupted by homologous recombination [12], HLA-A24 (A*2402) mice express a chimeric heavy chain and human β2m; the H-2Kb, H-2Db, and murine β2m genes were disrupted by homologous recombination (Lemonnier et al., unpublished), HLA-B7 (B*0702) mice express a chimeric heavy chain with the HLA-B*0702 α1 and α2 domains and the H-2Kd murine α3 domain [13]. The H-2Kb and H-2Db genes in HLA-B7 mice were inactivated by homologous recombination.

The HLA class II supertypes were DR2 (DRB1*1501), DR3 (DRB1*0301), and DR4 (DRB1*0401). The peptide-binding domain of HLA-DR2 transgenic mice is encoded by human sequences, while the membrane proximal portion containing the CD4-binding domain is encoded by mouse sequences (DRA1*0101: I-Eα and DRB1*1501: I-Eβ) [14]. HLA-DR3 transgenic mice express HLA-DRA*0101 and -DRB1*0301 [15]. HLA-DR4 transgenic mice express HLA-DRA*0101, -DRB1*0401, and human CD4 [16]. The derivation and validation of the above transgenic mice, which were bred onto C57BL/6 genetic background, had been described in the relevant publications.

### Immunization

Mice were immunized with the selected 196 peptides in 2 pools by use of a protocol which had been validated for T cell studies [17] and adapted for these transgenic mice studies. Peptides were pooled in matrixes as described [18] and injected in groups of 9 mice of each transgenic strain: two for matrix array screening, two for identifying individual peptides, four for characterizing apparent functional avidity of T cells to positive peptides at three titration points: 10, 1, and 0.1 μg/ml peptide concentrations, and one as a control (adjuvant alone). Mice were injected subcutaneously at the base of tail with 100 μl of the immunization peptide pool in TiterMax® Gold adjuvant (TiterMax, Norcross, Ga.) (1:1). The amount of each peptide injected was 1 μg/mouse. After two weeks, spleens were harvested for IFN-γ ELISpot assay.

### IFN-γ ELISpot Assay

Harvested spleens from immunized transgenic mice were selectively depleted of T cells by use of anti-CD8 or anti-CD4 antibody-coated immunomagnetic beads with LD columns (Miltenyi Biotec, Auburn, Calif.) according to the manufacturer's protocol. Flow cytometry analysis of the depleted cells indicated this method routinely achieved >95% depletion of the targeted cells. The resulting MHC class I or II depleted splenocytes were tested individually by IFN-γ ELISpot assays against the 196 influenza peptides arranged in two 10×10 matrix arrays, resulting in 40 peptide pools, where each peptide was present in two different pools, as described [18]. Peptides identified as immunogenic in the matrix array screen were retested individually in a confirmatory assay and a peptide titration assay. Thus, each ELISpot positive response was confirmed three times: by matrix array screening, individually by confirmatory assay and by peptide titration.

The ELISpot assays were performed using mouse IFN-γ ELISpot sets from BD Biosciences (San Jose, Calif.) according to the manufacturer's protocol. Briefly, the ELISpot plates were coated with anti-IFN-γ at 5 μg/ml and incubated at 4° C. overnight. The plates were blocked with RPMI 1640 containing 10% heat-inactivated fetal calf serum, 2 mM L-glutamine, 100 μg of streptomycin/ml, and 100 U of penicillin for 2 h at room temperature, and either CD8+- or CD4+-depleted splenocytes (0.5-1.0×10̂6 cells/well) were then added for assays of class II and I T cell epitopes, respectively. The cells were cultured at 37° C. in 5% CO2 in the presence of peptide pools (final concentration of each peptide was 10 μg/ml) or individual peptides at final concentrations of 10 μg/ml, 1 μg/ml, and 0.1 μg/ml. Wells with medium alone served as background; Concanavalin A (2.5 μg/ml; Sigma-Alrich, St. Louis, Mo.) was used as a polyclonal stimulator; and known HLA-restricted peptides from Dengue serotype 3 were included in each assay as positive controls. After 16 h of culture, the plates were washed and incubated with biotinylated anti-IFN-γ for 2 h at room temperature, followed by HRP-conjugated streptavidin for 1 h at room temperature. Reactions were developed with AEC substrate (Calbiochem-Novabiochem, San Diego, Calif.). Final enumeration of IFN-γ spot-forming cells (SFC) was performed using the Immunospot Series 3B Analyzer ELISPOT reader (Cellular Technologies, Shaker Heights, Ohio) with aid of the Immunospot software version 3.0 (Cellular Technologies), indicating the number of SFC/10̂6 cells. The results were considered positive if the number of SFC subtracted by those in the background (culture with medium alone) was above 10 and the number of SFC was higher than the background plus two standard deviations. The results shown are SFC minus background, which was consistently found to be less than 15 spots/10̂6 cells throughout the experiments.

### Presence of Experimentally Identified T Cell Epitopes in the Influenza a Highly Conserved Sequences

Published influenza T cell epitopes within the highly conserved sequences were identified by matching the curated T cell epitope sequences mapped in human from the Immune Epitope Database and Analysis Resource (IEDB, http://www.immuneepitope.org/) [19] with the highly conserved sequences. All these published epitope sequences were derived from various T cell assays that included T cell proliferation, IFN-γ ELISpot, HLA tetramer staining, and 51Cr release assays. Only epitope data from unique sequences and containing HLA restriction information were included.

### Determination of Human Self-Peptide in Influenza Peptides

The 196 influenza 17 aa peptides were compared using the blastp program against the non-redundant protein sequences database restricted to human (taxid:9606) at NCBI (http://ww.ncbi.nlm.nih.gov/BLAST/) to detect the presence of fragments identical to human peptides. As the default search parameters were not suitable to probe for short peptide sequences of length 30 or less, the following parameters were used: word size of 2, expectation value of 30,000, matrix was PAM30, low complexity filter was disabled, and composition-based statistics was set to ‘no adjustment’. We disregarded search results containing predicted sequences and human peptides with fewer than six contiguous identical residues as the probability of matching five or less residues is high and non-significant.

### Conservation and Variability of Influenza A(H1N1) T Cell Epitope Peptides

The dataset and methodology for identification of highly conserved influenza protein sequences among pathogenic influenza strains for the past 30 years had been described by Heiny et al. [11]. Briefly, 3763 NP, 3781 M1, 3111 PA, 3175 PB1, and 3144 PB2 sequences were extracted from the NCBI GenBank and GenPept databases (as of September 2006) and multiple sequence alignments of the individual proteins were performed. The Antigenic Variability Analyzer tool (AVANA) [20] was used to extract alignments of each 17 aa T cell epitope mapped in the transgenic mice and to identify the most frequent 17 aa sequence present in at least 80% of all recorded viruses. To compare 2007-2009 human H1N1 sequences with the T cell epitopes of A/New York/348/2003 (H1N1), aligned protein sequence records of human H1N1 M1, PB1, and PB2 retrieved from the NCBI Influenza Virus Sequence Database (http://www.ncbi.nlm.nih.gov/genomes/FLU/FLU.html, as of Jun. 17, 2009) were submitted into the AVANA tool to identify the most frequent sequence and its variants for each year.

### EXAMPLE 2

### Results

### Immunogenicity of Human and Avian Influenza A Highly Conserved Peptide Sequences

The previously described 54 highly conserved influenza A peptide sequences of 9 or more contiguous aa of the recorded human and avian influenza strains were represented by a total of 956 aa [11]. The majority of the conserved sequences, 650 aa, were in the PB1 and PB2 proteins; there were no conserved sequence in NA, M2, NS1, and NS2. A total of 196 peptides (BEI) of the A/New York/348/2003 (H1N1) M1, NP, PA, PB1, and PB2 proteins were selected based on the presence of the conserved sequences. The immunogenicity of these 196 conserved influenza peptides was studied by immunizing HLA-A2, -A24, -B7, -DR2, -DR3 and -DR4 transgenic mice. Organization of the 54 conserved sequences in the BEI 17 aa peptides depended on their length and position. Conserved sequences that spanned adjacent 17 aa peptides were repeated up to a maximum of 11 aa because of overlapping peptide synthesis (Table 1 and 2). Peptides with conserved sequences of less than 17 aa contained mixtures of conserved and non-conserved aa. Thirty-three (33) short conserved sequences (9 to 16 aa) were present in various lengths with adjacent non-conserved aa. Conserved sequences of greater length (22 sequences of 17 to 57 aa) were present as complete (65 of the 196 peptides) or partial sequences in the overlapping peptides. The longest conserved sequence was PB 1518-575 which was included as part of a cluster of completely conserved aa of 7 overlapping peptides.

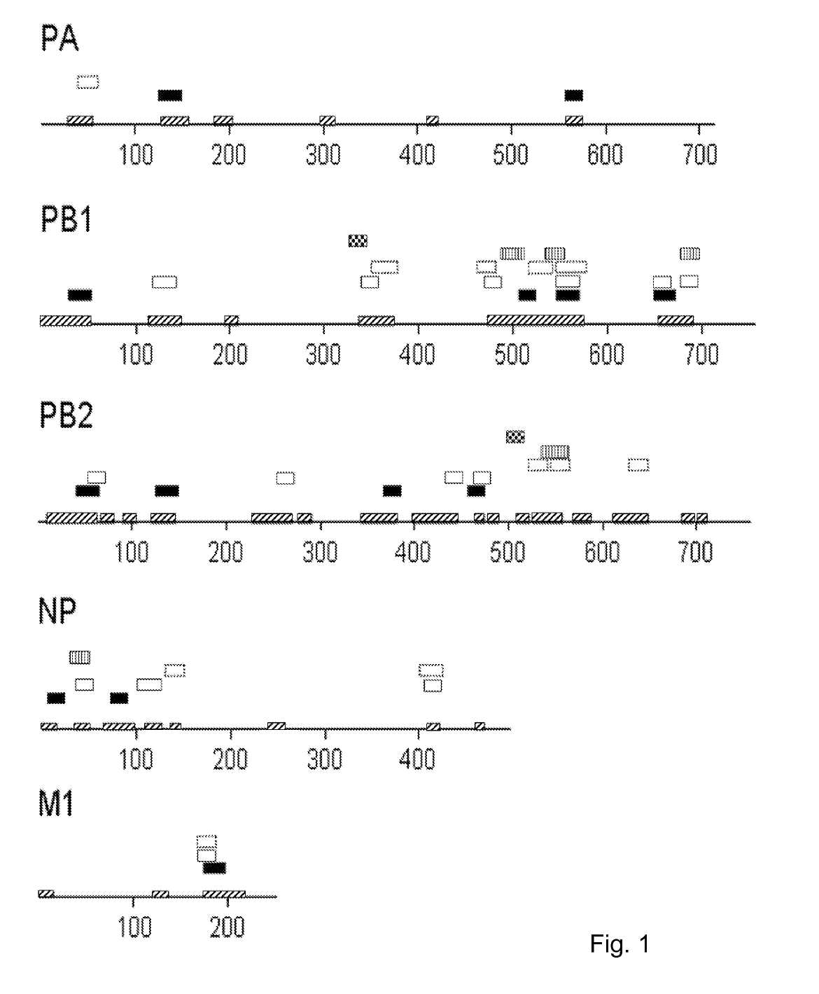

Immunization of the HLA transgenic mice with the 196 H1N1 peptides was carried out with 2 pools of about 100 peptides each, with groups of 9 mice of each transgenic strain. Interferon-γ (IFN-γ) ELISpot assays for HLA-restricted class I and class II responses were performed with splenocytes of the immunized mice that were depleted of CD4+ and CD8+ T cells, respectively, to identify the responding T cell subset. The initial assays contained matrix arrays of peptide pools followed by validation assays with individual peptides [18]. Of the 196 peptides, 54 contained T cell epitopes that elicited 63 ELISpot responses (8 A24, 2 B7, 16 DR2, 17 DR3, and 20 DR4) (Table 3). None of the 196 peptides tested induced T cell responses in mice expressing the HLA-A2 allele. Forty-seven (47) of the 54 epitope peptides were restricted by one HLA allele; eight class I and 39 class II. The remaining 7 peptides were presented by at least two HLA alleles of distinct supertypes i.e. they contained multiple or promiscuous T cell epitopes. PB1680-696 and PB2548-564 were presented by both HLA class I and II alleles. Sixteen (16) pairs of consecutive peptides were restricted by the same HLA allele, possibly because there were identical epitopes in the overlapping 11 aa sequence shared by the 2 adjacent peptides. Clusters of 2 or more T cell epitope peptides with at least 16 conserved aa were M1175-197, PB1120-142, 340-374, 489-576, and PB242-64, 126-146 (Table 3, FIG. 1).

TABLE 3
HLA-A24, -B7, -DR2, -DR3 and -DR4 restriction of 54 peptides of influenza proteins M1,
NP, PA, PB1 and PB2 that contain conserved sequences of 9 or more amino acids.

| Pro- | | | | | | |
|---|---|---|---|---|---|---|
| tein | ELISpot positive 17 aa peptide* | A24# | B7 | DR2 | DR3 | DR4 |
| M1 | 169 TNPLIRHENRMVLASTT 185 | — | — | 56 ± 5(0.1) | 120 ± 4(0.1) | — |
| | 175 HENRMVLASTTAKAMEQ 191 | — | — | — | — | 165 ± 1(0.1) |
| | 181 LASTTAKAMEQMAGSSE 197 | — | — | — | — | 115 ± 21(1) |
| NP | 7 KRSYEQMETDGERQNAT 23 | — | — | — | — | 52 ± 29(0.1) |
| | 31 RMIGGIGRFYIQMCTEL 47 | 45 ± 5 | — | — | — | — |
| | | (0.1) | | | | |
| | 37 GRFYIQMCTELKLNDYE 53 | — | — | — | 66 ± 7(1) | — |
| | 73 ERRNKYLEEHPSAGKDP 89 | — | — | — | — | 121 ± 1(0.1) |
| | 103 KWVRELVLYDKEEIRRI 119 | — | — | — | 614 ± 21(0.1) | — |
| | 109 VLYDKEEIRRIWRQANN 125 | — | — | — | 501 ± 42(0.1) | — |
| | 133 LTHIMIWHSNLNDTTYQ 149 | — | — | 238 ± 59(0.1) | — | — |
| | 402 SAGQISTQPTFSVQRNL 418 | — | — | 207 ± 3(0.1) | — | — |
| | 408 TQPTFSVQRNLPFDKTT 424 | — | — | 110 ± 14(1) | 41 ± 2(10) | — |
| PA | 42 LEVCFMYSDFHFINEQG 58 | — | — | 64 ± 11(1) | — | — |
| | 126 EVHIYYLEKANKIKSEK 142 | — | — | — | — | 37 ± 11(0.1) |
| | 132 LEKANKIKSEKTHIHIF 148 | — | — | — | — | 41 ± 10(0.1) |
| | 558 SRPMFLYVRTNGTSKIK 574 | — | — | — | — | 114 ± 24(0.1) |
| PB1 | 31 SHGTGTGYTMDTVNRTH 47 | — | — | — | — | 106 ± 1(0.1) |
| | 37 GYTMDTVNRTHQYSERG 53 | — | — | — | — | 125 ± 11(0.1) |
| | 120 DKLTQGRQTYDWTLNRN 136 | — | — | — | 142 ± 6(0.1) | — |
| | 126 RQTYDWTLNRNQPAATA 142 | — | — | — | 78 ± 0(0.1) | — |
| | 328 NQPEWFRNILSIAPIMF 344 | — | 60 ± 8 | — | — | — |
| | | | (10) | | | |
| | 340 APIMFSNKMARLGKGYM 356 | — | — | — | 175 ± 0(0.1) | — |
| | 352 GKGYMFESKSMKLRTQI 368 | — | — | 52 ± 2(1) | — | — |
| | 358 ESKSMKLRTQIPAEMLA 374 | — | — | 84 ± 20(0.1) | — | — |
| | 465 RFYRTCKLLGINMSKKK 481 | — | — | 231 ± 73(1) | — | — |
| | 471 KLLGINMSKKKSYINRT 487 | — | — | — | 116 ± 10(0.1) | — |
| | 489 TFEFTSFFYRYGFVANF 505 | 213 ± 9 | — | — | — | — |
| | | (0.1) | | | | |
| | 495 FFYRYGFVANFSMELPS 511 | 210 ± 25 | — | — | — | — |
| | | (0.1) | | | | |
| | 507 MELPSFGVSGVNESADM 523 | — | — | — | — | 274 ± 15(0.1) |
| | 519 ESADMSIGVTVIKNNMI 535 | — | — | 75 ± 10(0.1) | — | — |
| | 525 IGVTVIKNNMINNDLGP 541 | — | — | 159 ± 53(0.1) | — | — |
| | 537 NDLGPATAQMALQLFIK 553 | 92 ± 2(1) | — | — | — | — |
| | 548 LQLFIKDYRYTYRCHRG 564 | — | — | 61 ± 2(1) | 230 ± 23(0.1) | 97 ± 30(0.1) |
| | 554 DYRYTYRCHRGDTQIQT 570 | — | — | 109 ± 13(1) | 166 ± 22(0.1) | 76 ± 2(0.1) |
| | 560 RCHRGDTQIQTRRSFEI 576 | — | — | 194 ± 47(0.1) | — | — |
| | 650 GPAKNMEYDAVATTHSW 666 | — | — | — | 142 ± 45(0.1) | 41 ± 9(0.1) |
| | 656 EYDAVATTHSWVPKRNR 672 | — | — | — | — | 59 ± 2(0.1) |
| | 680 RGILEDEQMYQRCCNLF 696 | 78 ± 4 | — | — | 181 ± 10(0.1) | — |
| | | (0.1) | | | | |
| PB2 | 42 NPSLRMKWMMAMKYPIT 58 | — | — | — | — | 166 ± 3(0.1) |
| | 48 KWMMAMKYPITADKRIT 64 | — | — | — | — | 161 ± 18(0.1) |
| | 54 KYPITADKRITEMIPER 70 | — | — | — | 499 ± 4(0.1) | — |
| | 126 KHGTFGPVHFRNQVKIR 142 | — | — | — | — | 316 ± 20(0.1) |
| | 132 PVHFRNQVKIRRRVDIN 148 | — | — | — | — | 311 ± 37(0.1) |
| | 256 DQSLIIAARNIVRRAAV 272 | — | — | — | 169 ± 12(0.1) | — |
| | 369 RATAILRKATRRLIQLI 385 | — | — | — | — | 54 ± 2(0.1) |
| | 434 LLRHFQKDAKVLFLNWG 450 | — | — | — | 444 ± 14(0.1) | — |
| | 458 MGMIGILPDMTPSTEMS 474 | — | — | — | — | 238 ± 5(0.1) |
| | 464 LPDMTPSTEMSMRGVRV 480 | — | — | — | 324 ± 28(0.1) | — |
| | 500 RFLRVRDQRGNVLLSPE 516 | — | 184 ± 3 | — | — | — |
| | | | (0.1) | | | |
| | 524 TEKLTITYSSSMMWEIN 540 | — | — | 151 ± 67(0.1) | — | — |
| | 536 MWEINGPESVLINTYQW 552 | 289 ± 16 | — | — | — | — |
| | | (0.1) | | | | |
| | 542 PESVLINTYQWIIRNWE 558 | 226 ± 5 | — | — | — | — |
| | | (0.1) | | | | |
| | 548 NTYQWIIRNWETVKIQW 564 | 322 ± 44 | — | 96 ± 9(0.1) | — | — |
| | | (0.1) | | | | |
| | 630 RMQFSSLTVNVRGSGMR 646 | — | — | 104 ± 16(0.1) | — | — |
| | ELISpot responses | 8 | 2 | 16 | 17 | 20 |

*Conserved aa are in boldface. Consecutive peptides overlapping by 11 aa are aligned.
#Numbers are representative average IFN-γ spots forming cells per million splenocytes of individual transgenic mice that were positive at 10 μg/ml of peptide concentration. Number (10, 1 or 0.1) in parenthesis represents the lowest concentration of peptide (μg/ml) giving positive ELISpot response in peptide titration.
— represents no positive ELISpot response.

The apparent functional avidity of T cells to each of the 54 peptides was titrated at three peptide concentrations of 10, 1 and 0.1 μg/ml in IFN-γ ELISpot assays. Of the 63 positive ELISpot responses, including the responses of peptides restricted by multiple HLA alleles, 52 activated IFN-γ secretion at each of the three concentrations used in the ELISpot assay, 9 elicited at concentrations of 10 and 1 μg/ml, and 2 peptides (NP408-424 and PB1328-344) elicited solely at the highest peptide concentration (Table 3).

### EXAMPLE 3

### Presence of Reported T Cell Epitopes in the Conserved Sequences of Influenza A

The conserved peptides of this study were compared with reported T cell epitope sequences of humans infected with influenza A viruses extracted from the IEDB. Twenty-one (21) of about 800 reported human T cell epitopes of PB2, PB1, PA, NP, and M1 were found to contain sequences of 9 or more conserved amino acids of all recorded 1977-2006 influenza A viruses (Table 4). These were mainly from H1N1, H3N2, and H5N1 infections and included sequences restricted by a broad range of HLA class I and II alleles, including many not covered by the transgenic mice of this study. For example, the same T cell epitope “RMVLASTTAK” in M1178-187 was reported to be restricted by HLA-A3 and -A11 [21,22]. Clusters of overlapping epitopes were also observed within the conserved sequences, for example, M1123-137 had three overlapping epitopes (123 ALASCMGLIY 132 was restricted by A1; 125 ASCMGLIY 132 by B35; and 129 GLIYNRMGA 137 by A2) [21,23]. Thus, the highly conserved sequences contained common epitopes shared by pathogenic influenza strains and could be restricted by a broad range of HLA alleles.

TABLE 4
Presence of reported human influenza A T cell epitopes in 21 highly conserved aa
peptides of A/New York/348/2003 (H1N1).

| | | | | HLA allele | Published | |
|---|---|---|---|---|---|---|
| Highly conserved 17 aa eptide* | | | | this work^{#} | HLA alleles | Influenza strain |
| M1 | 1 | M [symbol] SIVPS | 17 | — | A2 | A/Puerto Rico/8/34 (H1N1) |
| M1 | 121 | A [symbol] A | 137 | — | A1, A2, B35, | A/Vietnam/1203/2004 (H5N1), Influenza |
| | | | | | DRB1*0404 | A (H3N2) |
| M1 | 169 | TNPLIR [symbol] | 185 | DR2, DR3 | B39, DR2, DRB1*0103, | A/Vietnam/1203/2004 (H5N1), Influenza |
| | | | | | DRB1*1101, | A |
| | | | | | DRB1*0701, DRB5*0101 | |
| M1 | 175 | [symbol] | 191 | DR4 | A3, A11, DRB1*0701 | A/Puerto Rico/8/34 (H1N1), |
| | | | | | | A/Vietnam/1203/2004 (H5N1) |
| NP | 61 | LTIER [symbol] K | 77 | — | A3 | Influenza A |
| NP | 67 | VLSAFDERRNKYLEEHP | 83 | — | DRB1*0101 | A/Vietnam/1203/2004 (H5N1) |
| NP | 73 | ERRNKYLEEHPSAGKDP | 89 | DR4 | DR1, DRB1*0101 | A/NT/60/68 (H3N2), A/Vietnam/1203/2004 |
| | | | | | | (H5N1) |
| NP | 91 | [symbol] KRVDGKWVRE | 107 | DR3 | A68 | A/Texas/1/77 (H3N2) |
| NP | 109 | VLYDKEEIRRIWRQANN | 125 | DR3 | DRB1*1101 | A/Vietnam/1203/2004 (H5N1) |
| NP | 402 | SAGQISTQPTFSVQRNL | 418 | DR2 | DRB1*0101, | A/Vietnam/1203/2004 (H5N1) |
| | | | | | DRB1*0404 | |
| PA | 42 | [symbol] NEQG | 58 | DR2 | A2 | A/Puerto Rico/8/34 (H1N1) |
| PB1 | 1 | [symbol] | 17 | — | A2 | Influenza A |
| PB1 | 37 | [symbol] RG | 53 | DR4 | A26 | Influenza A |
| PB1 | 346 | [symbol] | 362 | — | B62, B27 | Influenza A |
| PB1 | 352 | GKGYM [symbol] S [symbol] RTQI | 368 | DR2 | B44 | Influenza A |
| PB1 | 489 | [symbol] RYGFVANF | 505 | A24 | A1, B44 | Influenza A |
| PB1 | 501 | [symbol] VSGV | 517 | — | A2 | Influenza A |
| PB1 | 537 | NDL [symbol] QLFIK | 553 | A24 | B7 | Influenza A |
| PB1 | 560 | RCHRGD [symbol] EI | 576 | DR2 | B62 | Influenza A |
| PB1 | 566 | TQIQT [symbol] KKLWDQ | 582 | — | B27 | Influenza A (H3N2) |
| PB2 | 48 | K [symbol] TADKRIT | 64 | DR4 | A2 | A/Puerto Rico/8/34 (H1N1) |

*Conserved aa are in boldface. Published HLA epitopes were extracted from the IEDB. HLA class I epitopes are underlined and the first amino acid of each identified allele is italicized. HLA class II epitopes longer than 17aa are represented only by the corresponding residues in the 17aa peptides of A/New York/348/2003 (H1N1).
^{#}—represents no positive ELISpot response.

### EXAMPLE 4

### Analysis of the Presence of Human aa Sequences in Influenza Peptides

Each of the 196 influenza 17 aa peptides used in this study was compared with the human proteome sequences to investigate the possibility of human antigens that could trigger an autoimmune response to immunization. Specifically, we screened for exactly identical sequences of at least 8 continuous aa, which is the minimum binding peptide length for MHC class I [24]. Many of the conserved sequences of the influenza peptides contained sequences of 6 aa found in human proteins such as voltage-gated sodium channel, dystrophin etc. The longest influenza A sequence with an identical human counterpart was 7 aa of PA131-137 but none contained sequences of 8 or more aa identical to the human proteome.

TABLE 5
Determination of human self-peptides in representative influenza 17aa peptides.

| Viral peptide* | | | | Human peptide | Human protein name | GenPept ID |
|---|---|---|---|---|---|---|
| M1 | 169 | TNPLIRHENR [symbol] T | 185 | 26 MVLAST 31 | Ring finger protein 220 | NP_060620 |
| M1 | 175 | HENRMVLAST [symbol] Q | 191 | 140 TAKAME 145 | Mediator of cell motility 1 | NP_057039 |
| M1 | 181 | LASTTAKAM [symbol] SSE | 197 | 1387 EQMAGS 1392 | MYST histone acetyltransferase 3 | NP_001092882 |
| NP | 7 | [symbol] TDGERQNAT | 23 | 582 KRSYEQ 587 | Metastasis associated protein | NP_004680 |
| NP | 103 | KWVRELVLYDK [symbol] | 119 | 121 EEIRRI 126 | Annexin IV | NP_001144 |
| NP | 402 | SAGQISTQ [symbol] | 418 | 80 PTFSVQ 85 | Mucin 6, gastric | NP_005952 |
| NP | 408 | T [symbol] DKTT | 424 | 1805 QPTFSV 1810 | Chromodomain helicase DNA binding | NP_079410 |
| | | | | | protein 9 | |
| PA | 126 | EVHIY [symbol] K | 142* | 1266 YLEKANK 1272 | Dystrophin Dp427c isoform | NP_000100 |
| | | | | 1274 YLEKANK 1280 | Dystrophin Dp427m isoform | NP_003997 |
| | | | | 1151 YLEKANK 1157 | Dystrophin Dp427l isoform | NP_003998 |
| | | | | 1270 YLEKANK 1276 | Dystrophin Dp427pl isoform | NP_004000 |
| PB1 | 31 | SHGTGT [symbol] NRTH | 47 | 3151 GYTMDT 3156 | Polydom | NP_699197 |
| PB1 | 31 | SHGTG [symbol] TVNRTH | 47 | 2141 TGYTMD 2146 | Multiple EGF-like-domains 8 | NP_001401 |
| PB1 | 471 | KLLGIN [symbol] YINRT | 487 | 609 MSKKKS 614 | Suppressor variegation 4-20 homolog 1 | NP_060105 |
| | | | | | isoform 1 | |
| PB1 | 489 | TFEFT [symbol] GFVANF | 505 | 561 SFFYRY 566 | Phosphatidylinositol glycan anchor | NP_036459 |
| | | | | | biosynthesis | |
| PB1 | 537 | NDLG [symbol] QLFIK | 553 | 919 PATAQM 924 | Rho GTPase-activating protein | NP_055530 |
| PB1 | 548 | LQLFIK [symbol] RCHRG | 564 | 231 DYRYTY 236 | Syntaxin binding protein 5 isoform a | NP_640337 |
| PB2 | 256 | DQSLIIA [symbol] AV | 272 | 725 ARNIVR 730 | Akt substrate AS250 | NP_065076 |
| PB2 | 256 | DQSLI [symbol] AV | 272 | 1301 IAARNI 1306 | ATP-binding cassette, sub-family A, | NP_525023 |
| | | | | | member 6 | |
| PB2 | 458 | MGMIGILP [symbol] | 474 | 1964 DMTPST 1969 | Voltage-gated sodium channel Type II, | NP_066287 |
| | | | | | isoform 1 | |
| PB2 | 458 | MGMIGILP [symbol] | 474 | 1964 DMTPST 1969 | Voltage-gated sodium channel Type II, | NP_001035233 |
| | | | | | isoform 2 | |

*Conserved aa are in boldface. Italicized aa are found in human peptides. + PA131-137 shared 7aa identity with human Dystrophin Dp427 isoform proteins.

### EXAMPLE 5

### Variants of the Conserved T Cell Epitope Sequences

The 54 HLA-restricted T cell epitope peptides of A/New York/348/2003 (H1N1) strain were analyzed by the Antigenic Variability Analyzer (AVANA) tool for identification of (a) the consensus sequence (most frequent sequence) in the context of influenza A conserved sequences over the past 30 years, and (b) variants and percentage representation of 2007-2009 human H1N1 strains as compared to the 2003 H1N1 strain. Based on their conservation and variability, the 54 T cell epitope peptides formed three groups:

1) Seventeen (17) T cell epitope peptide sequences of the 2003 strain (11 PB1, 4 PB2, and 2 M1) had consensus sequences representing at least 88% and, for all but 2 consensus sequences represented at least 95% of all recorded human and avian influenza strains (Table 6A). In particular, PB1489-505 was 100% conserved in all H1N1 viruses. Several variant sequences within this group were recorded, but these were mostly single conservative amino acid substitutions representing a small fraction (less than 5%) of all the recorded 1977-2006 virus sequences. The major change in 2009 was the apparent complete replacement of 2 previous consensus sequences by variant sequences, each with 1 mutated aa (PB2132-148, 630-646).

TABLE 6(A)
Representation of 26 H1N1 T cell epitope peptide sequences among
all influenza A 1977-2003 strains and H1N1 strains 2007-2009.
A) 17 H1N1 sequences corresponding to the consensus sequences
with at least 88% representation. B) 9 sequences with single
amino acid substitutions from the consensus sequences
(≧80% representation).

| | | | | 1977-2006 | 2007 | 2008 | 2009 |
|---|---|---|---|---|---|---|---|
| | A/New York/348/2003 H1N1 | | | Influenza | human | human | human |
| Protein | ELISpot positive peptide§ | | | A* | H1N1″ | H1N1\hat{ } | H1N1+ |
| PB1 | 31 | SHGTGTGYTMDTVNRTH | 47 | 99 | 100 | 100 | 100 |
| | 120 | DKLTQGRQTYDWTLNRN | 136 | 97 | 100 | 100 | 100 |
| | 126 | RQTYDWTLNRNQPAATA | 142 | 99 | 100 | 100 | 100 |
| | 340 | APIMFSNKMARLGKGYM | 356 | 96 | 98 | 100 | 92 |
| | | -------------R--- | | 2 | 2 | — | 8 |
| | 489 | TFEFTSFFYRYGFVANF | 505 | 100 | 100 | 100 | 100 |
| | 495 | FFYRYGFVANFSMELPS | 511 | 99 | 100 | 100 | 100 |
| | 519 | ESADMSIGVTVIKNNMI | 535 | 97 | 100 | 100 | 99 |
| | | ----------------T | | # | — | — | 1 |
| | 525 | IGVTVIKNNMINNDLGP | 541 | 97 | 100 | 100 | 99 |
| | 537 | NDLGPATAQMALQLFIK | 553 | 98 | 100 | 100 | 99 |
| | | S---------------- | | 0.11 | — | — | 1 |
| | 548 | LQLFIKDYRYTYRCHRG | 564 | 98 | 100 | 100 | 100 |
| | 554 | DYRYTYRCHRGDTQIQT | 570 | 98 | 100 | 100 | 99 |
| | | ------------A---- | | 0.04 | — | — | 1 |
| PB2 | 126 | KHGTFGPVHFRNQVKIR | 142 | 96 | 96 | — | 98 |
| | | -Y--------------- | | # | — | — | 1 |
| | | ---S------------- | | # | — | — | 1 |
| | | -Q--------------- | | 0.14 | 3 | 100 | — |
| | 132 | PVHFRNQVKIRRRVDIN | 148 | 88 | 100 | 100 | — |
| | | ---------------T- | | 4 | — | — | 100 |
| | 500 | RFLRVRDQRGNVLLSPE | 516 | 92 | 100 | 100 | 100 |
| | 630 | RMQFSSLTVNVRGSGMR | 646 | 97 | 100 | 100 | — |
| | | ---------------L- | | 1 | — | — | 100 |
| M1 | 175 | HENRMVLASTTAKAMEQ | 191 | 98 | 100 | 100 | 100 |
| | 181 | LASTTAKAMEQMAGSSE | 197 | 95 | 100 | 100 | 100 |

§Highly conserved aa of 1977-2006 influenza A subtypes are in boldface.
*3175 PB1, 3144 PB2, and 3781 M1 human H1N1, H3N2, H1N2, H5N1, and avian H5N1 and other avian subtypes sequences circulating between 1977 and 2006 were extracted from NCBI GenBank and GenPept databases as of September 2006. Sequences representing less than 1% were not included unless they were also represented in the 2007-2009 strains.
All human PB1, PB2, and M1 H1N1 sequences from 2007 to 2009 were extracted from the Influenza Virus Resource on Jun 17, 2009.
+168 PB1, 171 PB2, and 280 M1 human H1N1 2009 sequences.
\hat{ }31 PB1, 31 PB2, and 39 M1 human H1N1 2008 sequences.
″314 PB1, 314 PB2, and 393 M1 human H1N1 2007 sequences.
#New sequence representation not found in the 1977-2006 influenza A subtypes sequences.

TABLE 6 (B)

| | A/New York/348/2003 | | | 1977-2006 | 2007 | 2008 | 2009 |
|---|---|---|---|---|---|---|---|
| | H1N1 ELISpot | | | Influenza | human | human | human |
| Protein | positive peptide§ | | | A* | H1N1″ | H1N1\hat{ } | H1N1+ |
| PB1 | | ---------------K- | | 86 | — | — | 99 |
| | 37 | GYTMDTVNRTHQYSERG | 53 | 13 | 99 | 84 | — |
| | | -----------R---K- | | # | — | — | 1 |
| | | -----------H----- | | # | — | 16 | — |
| | | ----------I------ | | 89 | 1 | — | — |
| | 507 | MELPSFGVSGVNESADM | 523 | 10 | 99 | 100 | 100 |
| | | ----------------L | | 86 | — | — | 99 |
| | 560 | RCHRGDTQIQTRRSFEI | 576 | 11 | 100 | 100 | — |
| | | ------A---------L | | 0.04 | — | — | 1 |
| | | ----S------------ | | 84 | — | — | 100 |
| | 650 | GPAKNMEYDAVATTHSW | 666 | 12 | 99 | 97 | — |
| | | -----I----------- | | 0.68 | — | 3 | — |
| | | ----T------------ | | 0.42 | 1 | — | — |
| | | -----------I----- | | 87 | — | — | 96 |
| | 656 | EYDAVATTHSWVPKRNR | 672 | 11 | 100 | 100 | — |
| | | -----------T----- | | 0.76 | — | — | 4 |
| | | -----------K----- | | 85 | — | — | 100 |
| | 680 | RGILEDEQMYQRCCNLF | 696 | 10 | 98 | 87 | — |
| | | --V-------------- | | 0.23 | 1 | 10 | — |
| | | ----------L------ | | # | — | 3 | — |
| PB2 | | -------------Q--- | | 89 | — | — | 100 |
| | 434 | LLRHFQKDAKVLFLNWG | 450 | 7 | 97 | 100 | — |
| | | ---------R------- | | 0.03 | 1 | — | — |
| | | ----------I------ | | 0.03 | 1 | — | — |
| | | -----------V----- | | 90 | — | — | 99 |
| | 536 | MWEINGPESVLINTYQW | 552 | 8 | 100 | 100 | 1 |
| | | -----V----------- | | 84 | — | — | 99 |
| | 542 | PESVLINTYQWIIRNWE | 558 | 8 | 99 | 100 | 1 |

2) A group of 9 PB1 and PB2 T cell epitope peptides of the New York/348/2003 H1N1 strain were variants of the 1977-2006 total recorded influenza A virus population at a single mutated aa position (Table 6B). These variant New York/348/2003 strain sequences represented less than 15% of the consensus sequences of the entire 1977-2006 avian and human virus population. One of these, PB1507-523, became the H1N1 consensus sequence of 2007-2009. For the others, a single aa modification to the BEI peptide would result in 96-100% representation in the 2009 human H1N1 population.

3) The remaining 28 peptides were each represented in the dataset by 2 to 7 variant sequences with multiple mutations (Table 7). The New York/348/2003 2003 sequences were the consensus form in only 13 of the 28 peptides and at reduced representations of 6 to 72% of the recorded viruses. As the variant forms contained a mixture of the conserved sequences and variable amino acids, it is not possible to predict the immunogenicity of the variant sequences represented in nature and their use as vaccine sequences. These data demonstrated that when T cell epitopes contain mixtures of conserved and non-conserved aa, the occurrences of mutated sequences in a subsequent influenza A strain are greatly enhanced.

TABLE 7
Representation of 28 (9 NP, 4 PA, 9 PB2,
5 PB1, and 1 M1) T cell epitope peptides
of A/New York/348/2003 (H1N1) among human
H1N1, H3N2, H1N2, H5N1, and other avian
subtypes circulating between 1977 to 2006.

| | A/New York/348/2003 | | | 1977-2006 |
|---|---|---|---|---|
| | H1N1 ELISpot | | | influenza |
| Protein | positive peptide§ | | | A* |
| NP | | ---------G------- | | 39 |
| | | -----------D----- | | 31 |
| | 7 | KRSYEQMETDGERQNAT | 23 | 22 |
| | | ---------G----D-- | | 3 |
| | | ---------S------- | | 1 |
| | | K-D-------------- | | 42 |
| | | --V-------------- | | 28 |
| | | --VS------------- | | 11 |
| | 31 | RMIGGIGRFYIQMCTEL | 47 | 8 |
| | | --V-------V------ | | 3 |
| | | K---------------- | | 2 |
| | | ---D------------- | | 2 |
| | | ---S------------- | | 2 |
| | | -------------S--- | | 75 |
| | | -------------S-H- | | 9 |
| | 37 | GRFYIQMCTELKLNDYE | 53 | 8 |
| | | -------------S-Q- | | 1 |
| | | ----V--------S--- | | 1 |
| | | -----------Q-S--- | | 1 |
| | | ----R------------ | | 49 |
| | 73 | ERRNKYLEEHPSAGKDP | 89 | 45 |
| | | ----R----N------- | | 2 |
| | | ------I---------- | | 24 |
| | | --M-------------- | | 22 |
| | | R-M-------------- | | 21 |
| | | --M---I---------- | | 16 |
| | 103 | KWVRELVLYDKEEIRRI | 119 | 7 |
| | | --M---I---------V | | 3 |
| | | --I---I---------- | | 2 |
| | | --M---I----D----- | | 1 |
| | 109 | VLYDKEEIRRIWRQANN | 125 | 50 |
| | | I---------------- | | 41 |
| | | I---------V------ | | 3 |
| | | I----D----------- | | 1 |
| | | ---L---------A--- | | 38 |
| | | ---M------------- | | 25 |
| | | ---M---------A--- | | 17 |
| | | -------------A--- | | 12 |
| | 133 | LTHIMIWHSNLNDTTYQ | 149 | 7 |
| | | ------V---------- | | 69 |
| | | ----T-V---------- | | 10 |
| | 402 | SAGQISTQPTFSVQRNL | 418 | 6 |
| | | ------I---------- | | 5 |
| | | ------V-A-------- | | 5 |
| | | ------V--------S- | | 3 |
| | | V------------E-S- | | 41 |
| | | V------------ERA- | | 35 |
| | 408 | TQPTFSVQRNLPFDKTT | 424 | 6 |
| | | I---------------- | | 3 |
| | | V--------S---ERA- | | 3 |
| | | V-A-----------P-- | | 2 |
| | | V------------ERS- | | 1 |
| PA | 42 | LEVCFMYSDFHFINEQG | 58 | 58 |
| | | -------------D-R- | | 27 |
| | | -------------D-RS | | 9 |
| | | -------------D--- | | 1 |
| | | ---------------R- | | 1 |
| | | --I----------D-R- | | 1 |
| | | ---------------L- | | 1 |
| | | ----------------N | | 47 |
| | 126 | EVHIYYLEKANKIKSEK | 142 | 37 |
| | | ---T------------- | | 9 |
| | | ----------------R | | 1 |
| | | -I--------------- | | 1 |
| | | ----------------E | | 1 |
| | | ---------S------- | | 1 |
| | | ----------N------ | | 47 |
| | 132 | LEKANKIKSEKTHIHIF | 148 | 47 |
| | | ----------R------ | | 2 |
| | | ----------E------ | | 1 |
| | | ---S------------- | | 1 |
| | 558 | SRPMFLYVRTNGTSKIK | 574 | 65 |
| | | ---------------V- | | 32 |
| PB2 | 42 | NPSLRMKWMMAMKYPIT | 58 | 60 |
| | | --A-------------- | | 39 |
| | 48 | KWMMAMKYPITADKRIT | 64 | 57 |
| | | ----------------M | | 28 |
| | | ----------------I | | 8 |
| | | --------------K-- | | 2 |
| | | -------------V--- | | 47 |
| | | ----------M------ | | 25 |
| | 54 | KYPITADKRITEMIPER | 70 | 9 |
| | | ----------I------ | | 7 |
| | | --------K-------- | | 2 |
| | | ----------MD----- | | 1 |
| | 256 | DQSLIIAARNIVRRAAV | 272 | 61 |
| | | ---------------T- | | 34 |
| | | ----V------------ | | 2 |
| | | ---------------I- | | 1 |
| | | -------------V--- | | 47 |
| | 369 | RATAILRKATRRLIQLI | 385 | 46 |
| | | ------------MI--- | | 3 |
| | 458 | MGMIGILPDMTPSTEMS | 474 | 43 |
| | | ---V-V----------- | | 39 |
| | | -----V----------- | | 5 |
| | | ---V------------- | | 4 |
| | | -------S--------- | | 1 |
| | | --------------I-- | | 46 |
| | | -----------L----- | | 25 |
| | | -----------L--I-- | | 10 |
| | 464 | LPDMTPSTEMSMRGVRV | 480 | 10 |
| | 524 | TEKLTITYSSSMMWEIN | 540 | 46 |
| | | --R-------------- | | 46 |
| | | M---------------- | | 3 |
| | | I-R-------------- | | 1 |
| | 548 | NTYQWIIRNWETVKIQW | 564 | 54 |
| | | -----------A----- | | 35 |
| | | -----V----------- | | 6 |
| | | -----------I----- | | 1 |
| PB1 | 328 | NQPEWFRNILSIAPIMF | 344 | 55 |
| | | --------V-------- | | 39 |
| | | K-------V-------- | | 1 |
| | | -----------M----- | | 1 |
| | 352 | GKGYMFESKSMKLRTQI | 368 | 47 |
| | | ---------R------- | | 47 |
| | | -R--------------- | | 2 |
| | | ----------------V | | 1 |
| | | ---------N------- | | 1 |
| | | --------R-------- | | 1 |
| | 358 | ESKSMKLRTQIPAEMLA | 374 | 47 |
| | | ---R------------- | | 46 |
| | | ----------V------ | | 1 |
| | | --R-------------- | | 1 |
| | | --------V-------- | | 75 |
| | | ----I---V-------- | | 13 |
| | 465 | RFYRTCKLLGINMSKKK | 481 | 10 |
| | | --V------------K- | | 46 |
| | | --V-------------- | | 43 |
| | 471 | KLLGINMSKKKSYINRT | 487 | 10 |
| M1 | 169 | TNPLIRHENRMVLASTT | 185 | 72 |
| | | -----K----------- | | 25 |
| | | ------------I---- | | 1 |

§Highly conserved aa are in boldface.
*3175 PB1, 3144 PB2, and 3781 M1 human H1N1, H3N2, H1N2, H5N1, and avian H5N1 and other avian subtypes sequences circulating between 1977 and 2006 were extracted from NCBI GenBank and GenPept databases as of September 2006. Sequences representing less than 1% of each dataset were excluded.

### REFERENCES

The disclosure of each reference cited is expressly incorporated herein.

1. Epstein S L, Kong W P, Misplon J A, Lo C Y, Tumpey T M, et al. (2005) Protection against multiple influenza A subtypes by vaccination with highly conserved nucleoprotein. Vaccine 23: 5404-5410.

2. Epstein S L, Tumpey T M, Misplon J A, Lo C Y, Cooper L A, et al. (2002) DNA vaccine expressing conserved influenza virus proteins protective against H5N1 challenge infection in mice. Emerg Infect Dis 8: 796-801.

3. Jimenez G S, Planchon R, Wei Q, Rusalov D, Geall A, et al. (2007) Vaxfectin-formulated influenza DNA vaccines encoding NP and M2 viral proteins protect mice against lethal viral challenge. Hum Vaccin 3: 157-164.

4. Powell T J, Strutt T, Reome J, Hollenbaugh J A, Roberts A D, et al. (2007) Priming with cold-adapted influenza A does not prevent infection but elicits long-lived protection against supralethal challenge with heterosubtypic virus. J Immunol 178: 1030-1038.

5. Epstein S L, Lo C Y, Misplon J A, Bennink J R (1998) Mechanism of protective immunity against influenza virus infection in mice without antibodies. J Immunol 160: 322-327.

6. Hamada H, Garcia-Hernandez Mde L, Reome J B, Misra S K, Strutt T M, et al. (2009) Tc17, a unique subset of CD8 T cells that can protect against lethal influenza challenge. J Immunol 182: 3469-3481.

7. Brown D M, Dilzer A M, Meents D L, Swain S L (2006) CD4 T cell-mediated protection from lethal influenza: perforin and antibody-mediated mechanisms give a one-two punch. J Immunol 177: 2888-2898.

8. Mozdzanowska K, Furchner M, Zharikova D, Feng J, Gerhard W (2005) Roles of CD4+ T-cell-independent and -dependent antibody responses in the control of influenza virus infection: evidence for noncognate CD4+ T-cell activities that enhance the therapeutic activity of antiviral antibodies. J Virol 79: 5943-5951.

9. Strutt T M, McKinstry K K, Swain S L (2009) Functionally diverse subsets in CD4 T cell responses against influenza. J Clin Immunol 29: 145-150.

10. Bui H H, Peters B, Assarsson E, Mbawuike I, Sette A (2007) Ab and T cell epitopes of influenza A virus, knowledge and opportunities. Proc Natl Acad Sci USA 104: 246-251.

11. Heiny A T, Miotto O, Srinivasan K N, Khan A M, Zhang G L, et al. (2007) Evolutionarily conserved protein sequences of influenza a viruses, avian and human, as vaccine targets. PLoS ONE 2: e1190.

12. Pascolo S, Bervas N, Ure J M, Smith A G, Lemonnier F A, et al. (1997) HLA-A2.1-restricted education and cytolytic activity of CD8(+) T lymphocytes from beta2 microglobulin (beta2m) HLA-A2.1 monochain transgenic H-2Db beta2m double knockout mice. J Exp Med 185: 2043-2051.

13. Rohrlich P S, Cardinaud S, Firat H, Lamari M, Briand P, et al. (2003) HLA-B*0702 transgenic, H-2KbDb double-knockout mice: phenotypical and functional characterization in response to influenza virus. Int Immunol 15: 765-772.

14. Vandenbark A A, Rich C, Mooney J, Zamora A, Wang C, et al. (2003) Recombinant TCR ligand induces tolerance to myelin oligodendrocyte glycoprotein 35-55 peptide and reverses clinical and histological signs of chronic experimental autoimmune encephalomyelitis in HLA-DR2 transgenic mice. J Immunol 171: 127-133.

15. Strauss G, Vignali D A, Schonrich G, Hammerling G J (1994) Negative and positive selection by HLA-DR3(DRw17) molecules in transgenic mice. Immunogenetics 40: 104-108.

16. Fugger L, Michie S A, Rulifson I, Lock C B, McDevitt G S (1994) Expression of HLA-DR4 and human CD4 transgenes in mice determines the variable region beta-chain T-cell repertoire and mediates an HLA-DR-restricted immune response. Proc Natl Acad Sci USA 91: 6151-6155.

17. Maciel M, Jr., Kellathur S N, Chikhlikar P, Dhalia R, Sidney J, et al. (2008) Comprehensive analysis of T cell epitope discovery strategies using 17DD yellow fever virus structural proteins and BALB/c (H2d) mice model. Virology 378: 105-117.

18. Tobery T W, Wang S, Wang X M, Neeper M P, Jansen K U, et al. (2001) A simple and efficient method for the monitoring of antigen-specific T cell responses using peptide pool arrays in a modified ELISpot assay. J Immunol Methods 254: 59-66.

19. Peters B, Sidney J, Bourne P, Bui H H, Buus S, et al. (2005) The immune epitope database and analysis resource: from vision to blueprint. PLoS Biol 3: e91.

20. Miotto O, Heiny A, Tan T W, August J T, Brusic V (2008) Identification of human-to-human transmissibility factors in PB2 proteins of influenza A by large-scale mutual information analysis. BMC Bioinformatics 9 Suppl 1: S18.

21. Assarsson E, Bui H H, Sidney J, Zhang Q, Glenn J, et al. (2008) Immunomic analysis of the repertoire of T-cell specificities for influenza A virus in humans. J Virol 82: 12241-12251.

22. Gianfrani C, Oseroff C, Sidney J, Chesnut R W, Sette A (2000) Human memory CTL response specific for influenza A virus is broad and multispecific. Hum Immunol 61: 438-452.

23. Lalvani A, Dong T, Ogg G, Patham A A, Newell H, et al. (1997) Optimization of a peptide-based protocol employing IL-7 for in vitro restimulation of human cytotoxic T lymphocyte precursors. J Immunol Methods 210: 65-77.

24. Biddison W E, Martin R (2001) Peptide binding motifs for MHC class I and II molecules. Curr Protoc Immunol Appendix 1: Appendix 1I.

25. Lo C Y, Wu Z, Misplon J A, Price G E, Pappas C, et al. (2008) Comparison of vaccines for induction of heterosubtypic immunity to influenza A virus: cold-adapted vaccine versus DNA prime-adenovirus boost strategies. Vaccine 26: 2062-2072.

26. Kreijtz J H, de Mutsert G, van Baalen C A, Fouchier R A, Osterhaus A D, et al. (2008) Cross-recognition of avian H5N1 influenza virus by human cytotoxic T-lymphocyte populations directed to human influenza A virus. J Virol 82: 5161-5166.

27. Thomas P G, Keating R, Hulse-Post D J, Doherty P C (2006) Cell-mediated protection in influenza infection. Emerg Infect Dis 12: 48-54.

28. Tompkins S M, Zhao Z S, Lo C Y, Misplon J A, Liu T, et al. (2007) Matrix protein 2 vaccination and protection against influenza viruses, including subtype H5N1. Emerg Infect Dis 13: 426-435.

29. Fernandez-Sesma A, Marukian S, Ebersole B J, Kaminski D, Park M S, et al. (2006) Influenza virus evades innate and adaptive immunity via the NS1 protein. J Virol 80: 6295-6304.

30. Berkhoff E G, Boon A C, Nieuwkoop N J, Fouchier R A, Sintnicolaas K, et al. (2004) A mutation in the HLA-B*2705-restricted NP383-391 epitope affects the human influenza A virus-specific cytotoxic T-lymphocyte response in vitro. J Virol 78: 5216-5222.

31. Carson R T, Desai D D, Vignali K M, Vignali D A (1999) Immunoregulation of Th cells by naturally processed peptide antagonists. J Immunol 162: 1-4.

32. Klenerman P, Zinkernagel R M (1998) Original antigenic sin impairs cytotoxic T lymphocyte responses to viruses bearing variant epitopes. Nature 394: 482-485.

33. Li X, Li R, Li Z (2006) Influenza virus haemagglutinin-derived peptides inhibit T-cell activation induced by HLA-DR4/1 specific peptides in rheumatoid arthritis. Clin Exp Rheumatol 24: 148-154.

34. Mirshahidi S, Ferris L C, Sadegh-Nasseri S (2004) The magnitude of TCR engagement is a critical predictor of T cell anergy or activation. J Immunol 172: 5346-5355.

35. Sloan-Lancaster J, Allen P M (1996) Altered peptide ligand-induced partial T cell activation: molecular mechanisms and role in T cell biology. Annu Rev Immunol 14: 1-27.

36. Tsitoura D C, Holter W, Cerwenka A, Gelder C M, Lamb J R (1996) Induction of anergy in human T helper 0 cells by stimulation with altered T cell antigen receptor ligands. J Immunol 156: 2801-2808.

37. Frahm N, Yusim K, Suscovich T J, Adams S, Sidney J, et al. (2007) Extensive HLA class I allele promiscuity among viral CTL epitopes. Eur J Immunol 37: 2419-2433.

38. Richards K A, Chaves F A, Sant A J (2009) Infection of HLA-DR1 transgenic mice with a human isolate of influenza a virus (H1N1) primes a diverse CD4 T-cell repertoire that includes CD4 T cells with heterosubtypic cross-reactivity to avian (H5N1) influenza virus. J Virol 83: 6566-6577.

39. Lee L Y, Ha do L A, Simmons C, de Jong M D, Chau N V, et al. (2008) Memory T cells established by seasonal human influenza A infection cross-react with avian influenza A (H5N1) in healthy individuals. J Clin Invest 118: 3478-3490.

40. Sette A, Sidney J (1999) Nine major HLA class I supertypes account for the vast preponderance of HLA-A and -B polymorphism. Immunogenetics 50: 201-212.

41. Sidney J, Peters B, Frahm N, Brander C, Sette A (2008) HLA class I supertypes: a revised and updated classification. BMC Immunol 9: 1.

42. Doytchinova I A, Flower D R (2005) In silico identification of supertypes for class II MHCs. J Immunol 174: 7085-7095.

43. Niedermann G (2002) Immunological functions of the proteasome. Curr Top Microbiol Immunol 268: 91-136.

44. Le Gall S, Stamegna P, Walker B D (2007) Portable flanking sequences modulate CTL epitope processing. J Clin Invest 117: 3563-3575.

45. Alexander-Miller M A, Leggatt G R, Berzofsky J A (1996) Selective expansion of high- or low-avidity cytotoxic T lymphocytes and efficacy for adoptive immunotherapy. Proc Natl Acad Sci USA 93: 4102-4107.

46. Derby M, Alexander-Miller M, Tse R, Berzofsky J (2001) High-avidity CTL exploit two complementary mechanisms to provide better protection against viral infection than low-avidity CTL. J Immuno1166: 1690-1697.

47. Sedlik C, Dadaglio G, Saron M F, Deriaud E, Rojas M, et al. (2000) In vivo induction of a high-avidity, high-frequency cytotoxic T-lymphocyte response is associated with antiviral protective immunity. J Virol 74: 5769-5775.

48. Gonzalez S, Zurcher T, Ortin J (1996) Identification of two separate domains in the influenza virus PB1 protein involved in the interaction with the PB2 and PA subunits: a model for the viral RNA polymerase structure. Nucleic Acids Res 24: 4456-4463.

49. Poole E, Elton D, Medcalf L, Digard P (2004) Functional domains of the influenza A virus PB2 protein: identification of NP- and PB1-binding sites. Virology 321: 120-133.

1. A polypeptide comprising: (a) a LAMP-1 lumenal sequence comprising SEQ ID NO: 19; (b) one or more segments of one or more influenza A proteins, wherein said segments comprise at least 9 contiguous amino acid residues selected from SEQ ID NO: 1-15, wherein segments are linked together by 0-20 amino acid residues; and (c) a LAMP transmembrane and cytoplasmic tail comprising SEQ ID NO: 21, wherein the lumenal sequence is amino-terminal to the one or more segments of an influenza A protein which are amino-terminal to the LAMP transmembrane and cytoplasmic tail.

2. The polypeptide of claim 1 comprising at least 3 of said segments.

3. The polypeptide of claim 1 comprising at least 5 of said segments.

4. The polypeptide of claim 1 comprising at least 10 of said segments.

5. The polypeptide of claim 1 comprising at least 15 of said segments.

6. A composition comprising a mixture of at least two polypeptides according to claim 1.

7. The polypeptide of claim 1 comprising a segment selected from the group consisting of SEQ ID NO: 3, 4, 5, 6, 8, 11, and 12.

8. A polypeptide consisting of an amino acid sequence selected from the group consisting of SEQ ID NO: 3, 4, 5, 6, 8, 11, and 12.

9. A polypeptide which comprises less than a full-length PB1 or PB2 protein of influenza A virus comprising an amino acid sequence selected from the group consisting of SEQ ID NO: 3, 4, 5, 6, 8, 11, and 12.

10. The polypeptide of claim 9 which is less than 150 amino acid residues in length.

11. A composition comprising a mixture of at least two polypeptides according to claim 8.

12. A composition comprising a mixture of at least two polypeptides according to claim 9.

13. A polynucleotide encoding the polypeptide of claim 1.

14. The polynucleotide of claim 13 wherein the polypeptide comprises at least 3 of said segments.

15. The polynucleotide of claim 13 wherein the polypeptide comprises at least 5 of said segments.

16. The polynucleotide of claim 13 wherein the polypeptide comprises at least 10 of said segments.

17. The polynucleotide of claim 13 wherein the polypeptide comprises at least 15 of said segments.

18. The polynucleotide of any of claims 13 wherein codons encoding the polypeptide are optimized according to most frequent human codon usage.

19. A composition comprising a mixture of at least two polynucleotides according to claim 13.

20. A polynucleotide encoding the polypeptide of claim 8.

21. A polynucleotide encoding the polypeptide of claim 9.

22. A composition comprising a mixture of at least two polynucleotides according to claim 20.

23. A composition comprising a mixture of at least two polynucleotides according to claim 21.

24. A nucleic acid vector which comprises the polynucleotide of claim 13, 20, or 21.

25. The nucleic acid vector of claim 24 which is a DNA virus.

26. The nucleic acid vector of claim 24 which is a RNA virus.

27. The nucleic acid vector of claim 24 which is a plasmid.

28. A host cell which comprises a nucleic acid vector of claim 24.

29. A method of producing a polypeptide comprising, culturing a host cell according to claim 28 under conditions in which the host cell expresses the polypeptide.

30. The method of claim 29 further comprising, harvesting the peptide from the culture medium or host cells.

31. A method of producing a cellular vaccine comprising:
transfecting antigen presenting cells with a nucleic acid vector according to claim 24 whereby the antigen presenting cells express the polypeptide.

32. The method of claim 31 wherein the antigen presenting cells are dendritic cells.

33. A method of making a vaccine, comprising: mixing together the polypeptide of claim 1, 8, or 9 and an immune adjuvant.

34. A vaccine composition comprising the polypeptide of claim 1, 8, or 9.

35. A method of immunizing a human or other animal subject, comprising:
administering to the human or other animal subject a polypeptide of claim 1, 8, or 9 or a nucleic acid vector according to claim 24 or a host cell according to claim 28, in an amount effective to elicit influenza A-specific T cell activation.

36. The method of claim 35 further comprising administering to the subject a live or attenuated influenza A vaccine.

37. The method of claim 35 further comprising administering an immune adjuvant to the subject.

38. The method of claim 35 wherein the administration is oral, mucosal, or nasal.

39. The method of claim 35 wherein the administration is intramuscular, intravenous, intradermal, intranasal, subcutaneous, or via electroporation.
